(19) United States
(12) Patent Application Publication
(10) Pub. No.: US 2025/0270662 A1
(43) Pub. Date: Aug. 28, 2025

(54) METHOD FOR ENUMERATION AND PHYSICAL CHARACTERIZATION OF NANOPARTICLES

(71) Applicants: Jason P. Gleghorn, Lincoln University, PA (US); Saurabh MODI, Newark, DE (US); Jasmine SHIRAZI, Dallas, TX (US)

(72) Inventors: Jason P. Gleghorn, Lincoln University, PA (US); Saurabh MODI, Newark, DE (US); Jasmine SHIRAZI, Dallas, TX (US)

(21) Appl. No.: 18/561,575

(22) PCT Filed: May 20, 2022
(86) PCT No.: PCT/US2022/030191

§ 371 Date: Nov. 16, 2023

Related U.S. Application Data
(60) Provisional application No. 63/191,171, filed on May 20, 2021.

(51) Int. Cl. C12Q 1/70; C12Q 1/6851; G01N 33/569

(52) U.S. Cl. CPC C12Q 1/701; C12Q 1/6851; G01N 33/56983; G01N 2469/10

(57) ABSTRACT

The present invention provides a method for quantifying moving heterologous nanoparticles in a suspension by imaging. The quantifying method comprises acquiring at least one z-stack of images within the suspension; tracking the nanoparticles in the images to identify unique nanoparticles; and enumerating the unique nanoparticles. Also provided is a method for characterizing size distribution of moving heterologous nanoparticles in a suspension by imaging. The characterization method comprises acquiring time lapse images; tracking the nanoparticles in the images to identify unique nanoparticles; determining the locations of each of the unique particles; determining the size of each of the unique nanoparticles; and aggregating the sizes of the unique nanoparticles.

### CROSS-REFERENCE TO RELATED APPLICATION

This application claims priority to U.S. Provisional Application No. 63/191,171, filed May 20, 2021, and the contents of which are incorporated herein by reference in their entireties for all purposes.

### FIELD OF THE INVENTION

This invention relates generally to characterization of nanoparticles by imaging.

### BACKGROUND OF THE INVENTION

The enumeration and characterization of nano-scale particles, also known as nanoparticles, is important across various disciplines including nanomedicine and extracellular vesicle (EV) research. Though a variety of methods exist for nano-scale particle counting by imaging, there is often a tradeoff between resolution and throughput. For example, electron microscopy provides unrivaled size and ultrastructural information, but it is impractical for enumeration or for any type of high throughput studies. High throughput methods often require specialized equipment and rely on either knowledge of the particle size ahead of time or their monodispersity in the sample. For example, dynamic light scattering (DLS) measures the scattered light intensity of nanoparticle suspensions and can translate this into relative particle size. However, it lacks the resolution to characterize samples of polydisperse size and does not supply reliable concentration data without previous knowledge of particle characteristics. Flow cytometry, a common method for nano-scale particle enumeration, presents challenges in measuring particles below 500 nm in diameter and routinely results in multiple small vesicles being counted as a single event in what is called a “swarming effect”. This is largely because this method measures the scattered light and not the particle itself which makes light scattering-based methods especially unreliable when the nanoparticle suspension is polydisperse. High end cytometers are able to detect particles down to 100 nm and resolve between 100 nm and 200 nm particles using light scatter alone, but this capability relies on extensive hardware modifications. Furthermore, the composition of the particle is an important factor for light scattering-based detection. For example, a 200 nm polystyrene bead exhibits an amount of scattered light comparable to a 500 nm lipid microvesicle, meaning that a 500 nm detection limit for a polystyrene bead translates to a detection limit of over 1 μm for a lipid microvesicle in the same instrument. The potential for undercounting becomes clear when one considers that a large number of EV subpopulations are smaller than 500 nm. Furthermore, light scattering-based techniques are prone to overcounting EVs due to false positives in the form of lipoproteins and protein aggregate contaminants.

A variety of specialized methods for EV quantification or characterization, such as fluorescence correlation spectroscopy (FCS), have been developed. FCS, which has recently been used for EV enumeration and measurement, offers greater sensitivity and information density. It also offers greater sensitivity than light scattering-based systems because it can detect individual fluorescent molecules. However, it requires specialized equipment and expertise for proper deployment. Similarly, advances in fluorescent imaging have allowed for impressive EV tracking using 3D holography while still requiring sophisticated engineering adaptations and techniques.

A simpler nano-flow cytometer was developed to image lipid nanovesicle on the order of hundreds of nanometers using a microfluidic channel and an epifluorescent microscope. This system reliably counts monodisperse samples and determines size distribution based on fluorescence intensity compared to a calibrated sample using a custom-designed microfluidic device. ExoCounter detects antibody-labeled EVs directly from human sera, but offers no size information. Others have used PKH/antibody co-labeling to image immobilized EVs using confocal microscopy or stimulated emission depletion (STED) microscopy. However, PKH and other lipid-based labeling methods alter the measured size of EVs and label numerous contaminating lipoproteins.

Nanoparticle tracking analysis (NTA) has quickly emerged as a common method for determining the size and concentration of nanoparticles in a suspension. One of the shortcomings of NTA systems (such as the NanoSight) is an inability to accurately characterize heterogeneous mixtures such as those that may occur while troubleshooting nanoparticle synthesis methods or characterizing extracellular vesicles (EVs). NanoSight measurements are also very sensitive to user defined settings, especially in heterogeneous samples where different settings for the camera level and detection threshold yield wildly inconsistent results. The NanoSight also operates in a narrow concentration range because samples with low concentrations may not contain enough particles for a proper statistical count, and highly concentrated samples may be too dense for the camera to overcome the overlaying effect in which large particles may obscure smaller ones.

Due to the constraints of physics, including the diffraction limit of light microscopy (˜200 nm) and Brownian motion of objects, direct imaging of nanoscale particles in suspension is challenging. A method often used to overcome these difficulties is to immobilize the particle of interest on a surface prior to imaging with super-resolution microscopes. This strategy eliminates Brownian motion but requires sample processing and knowledge of the nanoparticle composition to develop surface treatments for nanoparticle localization. To circumvent this, a simple system for epifluorescent imaging of nano-scale particles in suspension that allows for single particle tracking to determine size information in a heterogeneous suspension was developed. Whereas other fluorescent imaging-based approaches often rely on confocal microscopy or specialized custom imaging equipment and expertise, this method is compatible with standard widefield epifluorescent microscopy and requires inexpensive materials, making it accessible to more users.

Characterizing fluorescent nano-scale particles by imaging typically requires sophisticated imaging platforms such as confocal microscopy. Most imaging experts are skeptical of using a standard epifluorescent microscope for the characterization of nano-scale objects.

There remains a need for imaging-based methods for determining quantity and size distribution of suspended moving heterologous nanoparticles.

### SUMMARY OF THE INVENTION

The present invention relates to methods for characterizing moving heterologous nanoparticles in a suspension by imaging.

The present invention provides a method for quantifying moving heterologous nanoparticles in a suspension by imaging. The quantification method comprises: (a) acquiring at least one z-stack of images within the suspension with an exposure time of 20-150 ms, wherein each z-stack of images consists of at least 5 images at an interval of 0.4-0.6 μm; (b) tracking the nanoparticles in the images acquired in step (a) through x-axis, y-axis and z-axis of the suspension to identify unique nanoparticles; and (c) enumerating the unique nanoparticles, wherein by the number of the nanoparticles in the suspension is obtained.

According the quantification method, step (b) may comprise: (i) identify non-overlapping nanoparticle tracks in each of the images acquired in step (a); (ii) defining an exclusion zone around each of the non-overlapping nanoparticle tracks identified in step (i) that is equal to 20-30 nm in radius; and (iii) excluding non-overlapping nanoparticle tracks that overlap with at least one of the exclusion zones defined in step (ii), whereby each remaining non-overlapping nanoparticle track corresponds to a unique nanoparticle.

The quantification method may further comprise determining the concentration of the nanoparticles in the suspension.

According the quantification method, the exposure time may be 20-40 ms.

The quantification method may further comprise measuring a temperature.

According the quantification method, the nanoparticles may be natural nanoparticles, synthetic nanoparticles, or a combination thereof. The nanoparticles may be virus like particles (VLPs), and the suspension may have a concentration of 10⁵-10⁹ VLPs/mL. The nanoparticles may be labeled with a fluorescent agent.

Where the nanoparticles carry nucleic acids labeled with an intercalating dye having a fluorescent intensity proportional to the amount of the nucleic acids, the quantification method may further comprise detecting the fluorescent intensity of the intercalating dye, and quantifying the amount of the nucleic acids based on the detected fluorescent intensity. The nanoparticles may be viruses. The nanoparticles may be extracellular vesicles.

Where the nanoparticles are viruses having a genome made of the nucleic acids labeled with an intercalating dye having a fluorescent intensity proportional to the amount of the nucleic acids, and the quantification method may further comprise detecting the fluorescent intensity of the intercalating dye, quantifying the amount of the nucleic acid based on the detected fluorescent intensity, and determining the size of the genome based on the amount of the nucleic acids and the number of the nanoparticles.

Where the nanoparticles comprise charged nanoparticles and non-charged nanoparticles, the quantification method may further comprise (d) placing the suspension in contact with a charged surface, whereby the charged nanoparticles are attached to the charged surface, and the non-charged nanoparticles remain in the suspension and are enumerated in step (c).

The quantification method may further comprise: (e) acquiring images of a plurality of fields of view across the charged surface with an exposure time of 20-1000 ms; (f) tracking the charged nanoparticles attached to the charged surface in the images acquired in step (e) to identify unique charged nanoparticles; and (g) enumerating the unique charged nanoparticles attached to the charged surface.

The quantification method may further comprise determining a percentage of the charged nanoparticles based on the total number of the charged nanoparticles and the non-charged nanoparticles.

The present invention also provides a method for characterizing size distribution of moving heterologous nanoparticles in a suspension by imaging. The characterization method comprises: (a) acquiring time lapse images at a minimum of 30 frames per second (fps) with an exposure time of 20-150 ms; (b) tracking the nanoparticles in the images acquired in step (a) through x-axis, y-axis and z-axis of the suspension to identify unique nanoparticles; (c) determining the locations of each of the unique particles; (d) determining the size of each of the unique nanoparticles based on the locations determined in step (c); and (e) aggregating the sizes of the unique nanoparticles in step (d), whereby the size distribution of the nanoparticles in the suspension is characterized.

According to the characterization method, step (b) may comprise: (i) identify non-overlapping nanoparticle tracks in each of the images acquired in step (a); (ii) defining an exclusion zone around each of the non-overlapping nanoparticle tracks identified in step (i) that is equal to 20-30 nm in radius; and (iii) excluding non-overlapping nanoparticle tracks that overlap with at least one exclusion zones defined in step (ii), whereby each remaining non-overlapping nanoparticle track corresponds to a unique nanoparticle.

According to the characterization method, step (c) may comprise: (iv) determining the location of each unique nanoparticle based on the average position in the x-axis, y-axis and z-axis of all points in the corresponding non-overlapping track.

According to the characterization method, step (d) may comprise: (v) determining diffusion coefficient (D) for each unique nanoparticle using Equation 1:

$${Δ}_{ms} = 4 D Δ t \qquad Equation 1$$

wherein Δₘₛ is a mean squared displacement and Δt is a duration of a frame; and (vi) determining a diameter (d) of each unique nanoparticle using Equation 2:

$$d = \frac{{k}_{b} T}{3 πη D} \qquad Equation 2$$

wherein D is diffusion coefficient, T is temperature, η is medium viscosity, and k_{b} is the Boltzmann constant.

According to the characterization method, the time lapse images may be acquired at an intensity of 5-25%. The time lapse images may be acquired at an exposure time of 20-40 ms. At least 30 frames of images may be acquired during each exposure time.

The characterization method may further comprise measuring temperature.

According to the characterization method, the nanoparticles may be natural nanoparticles, synthetic nanoparticles, or a combination thereof. The nanoparticles may be virus like particles (VLPs), and the suspension may have a concentration of 10⁵-10⁹ VLPs/mL. The nanoparticles may be labeled with a fluorescent agent. The nanoparticles may be viruses. The nanoparticles may be extracellular vesicles.

Where the nanoparticles carry nucleic acid labeled with an intercalating dye having a fluorescent intensity proportional to the amount of the nucleic acid, the characterization method may further comprise detecting the fluorescent intensity of the intercalating dye, and quantifying the amount of the nucleic acid based on the detected fluorescent intensity.

Where the nanoparticles are viruses having a genome made of the nucleic acid labeled with an intercalating dye having a fluorescent intensity proportional to the amount of the nucleic acid, the characterization method may further comprise detecting the fluorescent intensity of the intercalating dye, quantifying the amount of the nucleic acid based on the detected fluorescent intensity, and determining the size of the genome based on the amount of the nucleic acid and the number of the nanoparticles.

Where the nanoparticles comprise charged nanoparticles and non-charged nanoparticles, the characterization method may further comprise: (f) placing the suspension on a charged surface, whereby the charged nanoparticles are attached to the charged surface, and the non-charged nanoparticles remain in the suspension and characterized for size distribution in step (e).

The characterization method may further comprise: (g) acquiring images of a plurality of fields of view across the charged surface with an exposure time of 20-1000 ms; (h) tracking the charged nanoparticles in the images acquired in step (e) to identify unique charged nanoparticles; and (i) enumerating the unique charged nanoparticles.

### BRIEF DESCRIPTION OF THE DRAWINGS

FIG. 1 shows a simple method of imaging small particles in suspension. (A) Two coverslips and double-sided tape were used to fabricate the NanoVis for imaging nano-scale particles. The device can be loaded similarly to a hemocytometer. (B) The device can be constructed to contain multiple sample chambers.

FIG. 2 shows widefield epifluorescent imaging for spatiotemporal tracking of nano-scale particles. (A) Confocal and widefield microscopy were used to image 50 nm fluorescent beads in suspension. (B) Due to the constant motion of particles, widefield microscopy allows for spatiotemporal tracking as it accounts for multiple planes within a single field of view.

FIG. 3 shows enumeration of nano-scale particles in suspension. (A) Particles are tracked through a Z-stack to ensure that each particle is counted only once. If a particle consistently appears within an exclusion radius across multiple slices (Case I), then it is counted as a single object. If it disappears for one slice only to reappear in the next (Case II), then that is also considered a single particle. However, if the particle strays from the exclusion radius in a new slice (Case III), then the particle in that slice is considered a unique object. (B) 200 nm fluorescent beads and 50 nm fluorescent beads were resolved using widefield microscopy.

FIG. 4 shows visualization of beads at a wide range of dilutions and enumerated using the custom platform. It was determined that 15 fields of view were sufficient for accurate counts of (A) 50 nm beads and (B) 200 nm beads.

FIG. 5 shows electron microscopy used to determine actual bead size. (A) TEM of 50 nm beads reveals a modal diameter of 55 nm. (B) TEM of 200 nm beads revealed a modal diameter of 183 nm.

FIG. 6 shows a three-fold serial dilution carried out for (A) 50 nm particles and (B) 200 nm particles and particle counts decreased accordingly, revealing that NanoVis counts are accurate across a wide range of concentrations

FIG. 7 shows particle tracking for the determination of size information. The diffusion coefficient of a particle in a given medium can be calculated by tracking its movement over a given time

FIG. 8 shows size characterization of 50 nm beads. (A) NanoSight readings measured a modal diameter of 47 nm, whereas (B) the NanoVis measured a modal diameter of 84 nm, which (C) was adjusted to 54 nm based on a linear calibration.

FIG. 9 shows size characterization of 200 nm beads. (A) NanoSight measurements of 200 nm beads yielded a modal diameter of 125 nm, whereas (B) the NanoVis measured a modal diameter of 208 nm, which (C) was adjusted to 190 nm based on a linear calibration curve.

FIG. 10 shows tracking of polydisperse particles. Fluorescent imaging offers benefits over light scattering based techniques because it allows for easier analysis of mixed populations. 200 nm particles (thick arrows) and 50 nm particles (thin arrows) were resolved and imaged within the same field of view.

FIG. 11 shows sampling requirements for mixed samples. (A) Running average analysis was performed for the size distribution showing that 25-30 fields of view were sufficient for accurate size determination (B) Running average analysis of calculated concentration reveals that 35-40 fields of view are sufficient for accurate enumeration.

FIG. 12 shows size analysis of a mixed bead sample. (A) The NanoSight was unable to detect all of the 50 nm beads. (B) The NanoVis detected the bimodal population present in the 50 nm and 200 nm bead containing mixture.

FIG. 13 shows tracked and characterized extracellular vesicles (A) SEM of microvesicles (MVs) and exosomes showing heterogeneity of population. (B) EVs were stained with CFSE and imaged using epifluorescence.

FIG. 14 shows enumeration of EVs using NanoSight and NanoVis. Counts obtained using both methods were within an order of magnitude of one another.

FIG. 15 shows size characterization of EVs. (A) NanoVis detected a multimodal distribution ranging from the nano-scale to the micro-scale. (B) NanoSight detected a unimodal nano-scale population (mode=116 nm), while (C) SEM measurements showed a heterogenous population that more closely matches the NanoVis.

FIG. 16 shows a new method for virus enumeration that is comparable to the Anodisc. (A) The Anodisc is a well-established method for enumerating viruses and depends on filtering a volume of SYBR-stained virus through a ceramic disc. (B) The Virometer allows the user to load 5-10 μL of SYBR-stained virus which then adheres directly to the coverslip for quick and easy imaging.

FIG. 17 shows surface treatments customized based on a virus's charge. Poly-L-lysine (PLL) coating confers a positive charge to the glass, which allows cultured T7 (net negative charge) to adhere, whereas plasma-treatment leaves a negative charge allowing aquatic isolates to adhere.

FIG. 18 shows T7 adheres to a positively charged surface more efficiently than to a negatively charged surface. Viruses were present on the coverslip surface with no free virus detected in a PLL-treated virometer after 90 mins. Plasma treated coverslips showed viruses adhered to the coverslip and in free suspension. Surface counts reveal that PLL-treated Virometers are capturing more viruses and with less variation compared to plasma-treated. (n=3; mean±s.d.).

FIG. 19 shows cultured virus that are accurately enumerated on the Virometer as compared to other methods. (A) Running average across multiple fields of view was calculated to determine when counts stabilize (B) A range of dilutions of T7 was tested on the Virometer and compared to the expected concentration to determine the operating range of this method. (C) Measured T7 concentration was compared across Anodisc, Virometer, and dPCR, with dPCR serving as the benchmark. (n=3; mean±s.d.). T7 virus was isolated by Rachel Keown who also performed Anodisc and dPCR processing.

FIG. 20 shows that nanoSight analysis captures additional particles. (A) T7 counts obtained using the NanoSight far exceeded those obtained using other established methods. E. coli conditioned-media contains similar concentrations of detectable particles. (B) Virometer images of T7 include faint punctate signal that can be thresholded out. (C) E. coli-conditioned medium contains similar punctate signal but no discernible VLPs.

FIG. 21 shows that environmental samples can be enumerated on the Virometer as compared to other methods. (A) The necessary number of imaging fields was confirmed for environmental samples. (B) Viral concentrates across a range of bodies of water (bay, creek, and pond) were compared. (n=3 for Virometer; mean±s.d.) (C) Counts of viral concentrates from BATS acquired via Anodisc or Virometer were compared (n=1). Samples were isolated by Rachel Keown who also performed Anodisc processing.

### DETAILED DESCRIPTION OF THE INVENTION

The present invention provides method for characterizing heterologous nanoparticles in a suspension by imaging. The invention is based on the inventors' surprising discovery of 3D imaging-based methods for characterizing suspended nanoparticles (e.g., quantity and size) by taking advantage of what would normally be considered shortcomings of epifluorescent microscopes by detecting and extracting information from the averaged fluorescent pathlines, also known as tracks, left behind by the motion of the nanoparticles. In particular, the inventors have discovered a novel method to identify unique nanoparticles in a suspension of moving heterologous nanoparticles based on their spatial positions in the suspension collected by special imaging processes. Additionally, these methods enable the quantification of particle size distribution from a mixed polydisperse particle suspension. Further, the inventors have successfully separated a desirable population of nanoparticles from the suspension by attaching them onto a surface for two-dimensional (2D) imaging of the attached nanoparticles while performing 3D imaging of the nanoparticles remaining in the suspension.

The term “nanoparticles” as used herein refers to objectives having a diameter in the range of 1-1,000 nm. The nanoparticles may be natural nanoparticles, synthetic nanoparticles, or a combination thereof. For example, the nanoparticles may be biological objects such as microorganisms (e.g., viruses) or biologically derived nanoparticles, i.e., nanoparticles produced by biological organisms (e.g., extracellular vesicles).

The term “heterologous nanoparticles” as used herein refers to a mixture of nanoparticles having different sizes and/or charges.

The term “imaging” as used herein refers to a process of generating an image, also known as an imaging plane or slice, of objects (e.g., nanoparticles) in a space (e.g., suspension) by exposure of a detector to signals from the objects. The signal may be a fluorescent signal emitted by the objects. The detector is any device capable of capturing the signals. For example, the detector may be a microscope or a cell phone camera equipped with a magnification lens. The spatial locations of the objects in the space may be defined by coordinates along x-axis, y-axis, and z-axis of the space. The x-axis is an arbitrary direction within the 2D image plane. The y-axis is a direction orthogonal to the x-axis in the 2D imaging plane. The z-axis is a direction orthogonal to both the x-axis and y-axis and orthogonal to the 2D imaging focal plane. The image is a two-dimensional (2D) plane along the x-axis and the y-axis perpendicular to each other, and the z axis orthogonal to the 2D plane.

The term “exposure time” as used herein refers to the duration of exposure of a detector to signals from objects to generate an image of the objects. The exposure time may be adjusted to obtain the best resolution of an image of the objects. The exposure time may be adjusted to obtain an image providing desirable information (e.g., number or size) of the objects.

The term “intensity” as used herein refers to the strength of signals from objects to which a detector is exposed to generate an image of the objects. The intensity may be adjusted to obtain the best resolution of an image of the objects. The intensity may be adjusted to obtain an image providing desirable information (e.g., number or size) of the objects.

The term “z-stack of images” as used herein refers to a series of images, also known as imaging planes or slices, generated by exposure of a detector to signals from objects in a space while the detector is placed at different distances from the objects. The images are parallel 2D planes, each of which has an x-axis and a y-axis perpendicular to each other, and separated by intervals along a z direction orthogonal to the parallel 2D planes, i.e., z-axis. An interval is the distance between two sequential images. The intervals among the z-stack of images may be the same or different. The spatial location of objects in the z-stack of images may be defined by coordinates along the x-axis, y-axis and z-axis of the space. Each image in a z-stack is indexed by its z-position in the space.

The term “time lapse images” as used herein refers to a series of images, also known as imaging planes or slices, generated after exposure of a detector to signals from objects in a space at different time points, based on frames per second, while the detector is placed at the same distance from the objects. The image is a two-dimensional (2D) plane along the x-axis and the y-axis perpendicular to each other, and the z axis orthogonal to the 2D plane. Each imaging plane or slice has a z position. The spatial location of the objects in each image may be defined by coordinates along the x-axis, the y-axis, and the z-axis of the space. Each time lapse image is indexed to its time point.

The term “tracking” as used herein refers to following an object in a path as the object moves in a space over time. The path is also called the object's track. The track is an aggregate of spatial locations of the object at different time points. Each spatial location of the object is defined by coordinates along the x-axis, the y-axis, and the z-axis in the space.

The term “diffusion coefficient” as used herein refers to the amount of a nanoparticle that diffuses across a unit area in 1 s under the influence of a gradient of one unit.

The present invention provides a method for quantifying moving heterologous nanoparticles in a suspension by imaging. The quantification method comprises (a) acquiring at least one z-stack of images within the suspension; (b) tracking the nanoparticles in the acquired images through x-axis, y-axis and z-axis of the suspension to identify unique nanoparticles; and (c) enumerating the unique nanoparticles. As a result, the number of the nanoparticles in the suspension is obtained.

The z-stack of images may be acquired as fast as possible. The exposure time may be in the range from about 20 ms to about 150 ms. For example, the exposure time may be about 33, 50, 70 or 90 ms. The z-stack of images may be acquired at an intensity in the range from about 5 to about 30%. For example, the intensity may be about 10% or 20%.

Each z-stack of images may consist of at least about 3, 4, 5, 6, 7, 8, 9, 10, 11, 12, 13, 14, 15, 16, 17, 18, 19 or 20 images at an interval in the range from about 0.4 μm to about 0.6 μm. For example, each z-stack of images consists at least about 5 images at an interval of about 0.5 μm. As more z-stacks of images are acquired, the quantity of the nanoparticles becomes more accurate.

The nanoparticles in the suspension move over time and generate tracks. Some tracks may overlap with each other. The overlapping tracks may be merged to generate non-overlapping tracks.

According to the quantification method, the tracking step may comprise identify non-overlapping nanoparticle tracks in each of the acquired images; defining an exclusion zone around each of the identified non-overlapping nanoparticle tracks; and excluding non-overlapping nanoparticle tracks that overlap with at least one of the defined exclusion zones. As a result, each remaining non-overlapping nanoparticle track corresponds to a unique nanoparticle.

For each non-overlapping nanoparticle track, the exclusion zone may be enclosed by a line about at least 10, 15, 20, 25, 30, 35 or 40 nm, no more than at least about 10, 15, 20, 25, 30, 35 or 40 nm, or about 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40 or 30-35 nm, in radius from the non-overlapping nanoparticle track.

The quantification method may further comprise determining the concentration of the nanoparticles in the suspension. The concentration of the nanoparticles in the suspension may be calculated by dividing the number of the nanoparticles by the volume of the suspension.

The quantification method may further comprise measuring a temperature. The imaging process may be adjusted based on the measure temperature to improve the resolution of the acquired images.

According to the quantification method, the nanoparticles are natural nanoparticles, synthetic nanoparticles, or a combination thereof. The natural nanoparticles may be microorganisms (e.g., viruses) or biologically derived nanoparticles. The biologically derived nanoparticles may be extracellular vesicles or virus like particles (VLPs). The nanoparticles are virus like particles (VLPs), and the suspension may have a concentration of 10⁵-10⁹ VLPs per mL.

According to the quantification method, the nanoparticles may be labeled with a fluorescent agent. The fluorescent agent may be a dye, intercalating agent (e.g., 5-ethynyl-2′-deoxyuridine (EdU)), or exogenous label (e.g., antibody).

According to the quantification method, the nanoparticles may carry nucleic acids (e.g., RNA or DNA). The nucleic acid may be labeled with an intercalating dye, which has a fluorescent intensity proportional to the amount of the nucleic acids. The quantification method may further comprise detecting the fluorescent intensity of the intercalating dye, and quantifying the amount of the nucleic acids based on the detected fluorescent intensity.

In one embodiment, the nanoparticles are viruses having a genome made of the nucleic acids labeled with an intercalating dye, which has a fluorescent intensity proportional to the amount of the nucleic acids, the quantification method may further comprise detecting the fluorescent intensity of the intercalating dye, quantifying the amount of the nucleic acids based on the detected fluorescent intensity, and determining the size of the genome based on the amount of the nucleic acids and the number of the viruses. In another embodiment, the nanoparticles are extracellular vesicles.

Where the nanoparticles in the suspension comprise charged nanoparticles and non-charged nanoparticles, the quantification method may further comprise placing the suspension in contact with a charged surface. As a result, the charged nanoparticles are attached to the charged surface, and the non-charged nanoparticles remain in the suspension and are enumerated. The charged surface may be a glass surface.

After the charged nanoparticles are attached to the charged surface, the quantification method may further comprise acquiring images of a plurality of fields of view across the charged surface; tracking the charged nanoparticles attached to the charged surface in the acquired images to identify unique charged nanoparticles; and enumerating the unique charged nanoparticles attached to the charged surface. The exposure time may be in the range from about 20 ms to about 1,000 ms, for example, 33, 50, 70 or 90 ms. The unique charged nanoparticles may be identified using the tracking step of the quantification method.

Once the charged nanoparticles and the non-charged nanoparticle are enumerated, a percentage of the charged nanoparticles based on the total number of the charged nanoparticles and the non-charged nanoparticles may be determined. Similarly, a percentage of the non-charged nanoparticles based on the total number of the charged nanoparticles and the non-charged nanoparticles may be determined.

A method is provided for characterizing size distribution of heterologous nanoparticles in a suspension by imaging. The characterization method comprises (a) acquiring time lapse images; (b) tracking the nanoparticles in the acquired images through x-axis, y-axis and z-axis of the suspension to identify unique nanoparticles; (c) determining the locations of each of the unique particles; (d) determining the size of each of the unique nanoparticles based on the determined locations; and (e) aggregating the sizes of the unique nanoparticles. As a result, the size distribution of the nanoparticles in the suspension is characterized.

The time lapse images may be acquired at a minimum of about 20, 25, 30, 35, 40, 45 or 50 frames per second (fps). The exposure time may be in the range from about 20 ms to about 150 ms. The time lapse images may be acquired at an intensity of about 5-25%, for example, about 10% or 20%. The time lapse images may be acquired at an exposure time of about 20-40 ms, for example, about 33 ms.

The time lapse images may be acquired at least about 10, 20, 30, 40, 50, 100, 500, 1,000, 5,000, or 10,000 frames of images during each exposure time. The more frames of images are acquired, the more information of the nanoparticles is obtained, and more accurate nanoparticle information is obtained.

The tracking step in the characterization method may be the same as the tracking step in the quantification method. The nanoparticles in the suspension move over time and generate tracks. Some tracks may overlap with each other. The overlapping tracks may be merged to generate non-overlapping tracks.

According to the characterization method, the tracking step may comprise identify non-overlapping nanoparticle tracks in each of the acquired images; defining an exclusion zone around each of the identified non-overlapping nanoparticle tracks; and excluding non-overlapping nanoparticle tracks that overlap with at least one of the defined exclusion zones. As a result, each remaining non-overlapping nanoparticle track corresponds to a unique nanoparticle.

For each non-overlapping nanoparticle track, the exclusion zone may be enclosed by a line about at least about 10, 15, 20, 25, 30, 35 or 40 nm, no more than at least about 10, 15, 20, 25, 30, 35 or 40 nm, or about 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40 or 30-35 nm, in radius from the non-overlapping nanoparticle track.

According to the characterization method, the location of each unique nanoparticle may be determined based on the average position in the x-axis, y-axis and z-axis of all points in the corresponding non-overlapping track.

To determine the size of each of the unique nanoparticles, the diffusion coefficient (D) may be determined for each unique nanoparticle using Equation 1.

$${Δ}_{ms} = 4 D Δ t \qquad Equation 1$$

Δₘₛ is a mean squared displacement and Δt is a duration of a frame. Then, the diameter (d) of each unique nanoparticle may be determined using Equation 2.

$$d = \frac{{k}_{b} T}{3 πη D} \qquad Equation 2$$

D is diffusion coefficient. T is temperature. η is medium viscosity. k_{b} is the Boltzmann constant.

The characterization method may further comprise measuring a temperature. The imaging process may be adjusted based on the measure temperature to improve the resolution of the acquired images.

According to the characterization method, the nanoparticles are natural nanoparticles, synthetic nanoparticles, or a combination thereof. The natural nanoparticles may be microorganisms (e.g., viruses) or biologically derived nanoparticles. The biologically derived nanoparticles may be extracellular vesicles or virus like particles (VLPs). Where the nanoparticles are virus like particles (VLPs), and the suspension may have a concentration of 10⁵-10⁹ VLPs per mL.

According to the characterization method, the nanoparticles may be labeled with a fluorescent agent. The fluorescent agent may be a dye, intercalating agent (e.g., 5-ethynyl-2′-deoxyuridine (EdU)), or exogenous label (e.g., antibody).

According to the characterization method, the nanoparticles may carry nucleic acids (e.g., RNA or DNA). The nucleic acid may be labeled with an intercalating dye, which has a fluorescent intensity proportional to the amount of the nucleic acids. The characterization method may further comprise detecting the fluorescent intensity of the intercalating dye, and quantifying the amount of the nucleic acids based on the detected fluorescent intensity.

In one embodiment, the nanoparticles are viruses having a genome made of the nucleic acids labeled with an intercalating dye, which has a fluorescent intensity proportional to the amount of the nucleic acids, the characterization method may further comprise detecting the fluorescent intensity of the intercalating dye, quantifying the amount of the nucleic acids based on the detected fluorescent intensity, and determining the size of the genome based on the amount of the nucleic acids and the number of the viruses. In another embodiment, the nanoparticles are extracellular vesicles.

Where the nanoparticles in the suspension comprise charged nanoparticles and non-charged nanoparticles, the characterization method may further comprise placing the suspension in contact with a charged surface. As a result, the charged nanoparticles are attached to the charged surface, and the non-charged nanoparticles remain in the suspension and are characterized for size distribution. The charged surface may be a glass surface.

After the charged nanoparticles are attached to the charged surface, the characterization method may further comprise acquiring images of a plurality of fields of view across the charged surface; tracking the charged nanoparticles attached to the charged surface in the acquired images to identify unique charged nanoparticles; and enumerating the unique charged nanoparticles attached to the charged surface. The exposure time may be in the range from about 20 ms to about 1,000 ms, for example, 33, 50, 70 or 90 ms. The unique charged nanoparticles may be identified using the tracking step of the characterization method.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Device Fabrication

A multi-well chamber is created to contain individual samples within each chamber and a control/blank sample to serve as a background subtraction sample. In one embodiment, thin cut strips of double coated tape of defined and uniform thickness was placed on a clean microscope coverslip to form the sides of individual chambers. A clean glass coverslip was then adheaded to the other side of the tape, resulting in multiple chambers in a single device, with a glass coverslip bottom and top and tape sides.

### Example 2. Processing the Images to Get the Locations in X & Y of the Centroids of Each Object in Each Time Slice

Each time slice image is intensity adjusted by saturating the top 0.01% and the bottom 1% of pixel intensities. Then median filtering (medfilt2( )) is carried out using a 3×3 pixel neighborhood. To even out the background a top hat transform is applied with a 200 pixel sized disc element. Then the image is binarized according to a user defined quantile (around 90%) of pixel intensities. The binary image is then area opened (bwareaopen( ) cutoff=10 pixel) and then morphologically opened (imopen( ) using disc of size 3 pixels) to remove small objects. If there are holes in the binary mask, they are filled (using imfill( ). Smears (islands of connected white pixels) were identified using bwconncomp( ) and their major axis length was obtained using regionprops( ) Potential halos are identified as objects with major axis lengths larger than a user defined threshold. These objects were eroded using imerode( ) with a 20 pixel size disc element. This binary image is the mask used for object specific local thresholding of the original image after intensity adjustment. Using bwconncomp( ) and regionprops( ) we get the major axes and the centroids of the mask objects. Then using the intensities of pixels located <0.75× major axis length away from the centroid but excluding the pixels in the mask object itself, the threshold is set as: mean+ (min (major axis length/400,1)+1.5)*std. A new smaller mask object is generated within the previous one by binarizing intensities based on this threshold. The objects are filtered for solidity>0.3 and aspect ratio<3 using regionprops ( ) to remove halo fragments.

Some halos leave fragments, approximately equidistant from the center of the halo in the resulting mask, hence if the user sees large number of halos visually, they can enable the following dehaloing algorithm. Using pdist( ) the vector of Euclidean distances between centroids of each object are obtained. If more than two pairwise distances are within 10 pixels of each other, the corresponding objects are halo fragments and are deleted.

The final centroid locations of objects in the resulting binary image are stored along with the time slice number in a cell array.

### Example 3. Use of the Centroid Locations in X & Y to Track Objects in Different Time Slices and Estimate the Object Size Based on the Stokes-Einstein Equation

The script starts by loading the data file and corresponding matrix of X & Y locations of centroids of objects and time slice number. The trackmem( ) function tracks objects and takes this matrix as argument with “maxdisp” radius of exclusion/maximum displacement that objects can move in consecutive slices, “dim” no. dimensions of object movement, “goodenough” number of slices that objects need to persist within the radius of exclusion, “memory” is the max number of slices that objects in a track can be absent for, to resume the same track when they reappear within the radius of exclusion in subsequent slices. The squared displacement is calculated as: SD_{i,j}=Delta x_{i,j}\hat{ }2+Delta y_{i,j}\hat{ }2, where the Delta x and Delta y are the x & y displacement of the object in slice j with respect to slice j−1. The diffusion constant is calculated as: D_i=mean (SD_{i,j}/(4 Delta t)), with Delta t being the time difference between the two time slices j and j−1. The Einstein-Stokes equation is then applied to give the diameter of the object corresponding to track i as: d_i=k T/(3 pi D_i μ), with k being the Boltzmann's constant, T is temperature, μ is the dynamic viscosity. This gives us the distribution of sizes of objects in the sample. The modes of the distribution are linearly transformed according to the calibration for 50 nm beads and 200 nm beads.

### Example 4. Processing the Images to Obtain Particle Concentration by Counting the Number of Tracks/Objects Per Unit Volume

Then median filtering (medfilt2( )) is carried out using a 3×3 pixel neighborhood. To even out the background a top hat transform is applied with a 200-pixel sized disc element. Then the image is binarized according to a user defined quantile (around 90%) of pixel intensities. The binary image is then area opened (bwareaopen( ) cutoff=10 pixel) and then morphological closing is implemented with a disk element of radius 3 pixel. Potential halos are identified as objects with major axis lengths larger than a user defined threshold. These objects were eroded using imerode( ) with a 20-pixel size disc element. This binary image is the mask used for object specific local thresholding of the original image after intensity adjustment. Using bwconncomp( ) and regionprops( ) we get the major axes and the centroids of the mask objects. Then using the intensities of pixels located <0.75× major axis length away from the centroid but excluding the pixels in the mask object itself, the threshold is set as: mean+(min (major axis length/400,1)+1.5)*std. A new smaller mask object is generated within the previous one by binarizing intensities based on this threshold. The objects are filtered for solidity>0.3 and aspect ratio<3 using regionprops( ) to remove halo fragments. The final centroid locations of objects in the resulting binary image are stored along with the time slice number in a matrix.

After the locations of centroids of objects and time slice number are determined. The first and last slices are deleted as they can have image artifacts. The trackmem( ) function tracks objects and takes this matrix as argument with “maxdisp” radius of exclusion/maximum displacement that objects can move in consecutive slices, “dim” no. dimensions of object movement, “goodenough” number of slices that objects need to persist within the radius of exclusion, “memory” is the max number of slices that objects in a track can be absent for, to resume the same track when they reappear within the radius of exclusion in subsequent slices. The cumulative number of tracks are counted as a function of Z position of the slice in each scene and the following formula is applied to get concentration=Cumulative no. tracks/(No. x pixels×No. y pixels×pixel size\hat{ }2×no. z slices×Delta z). The concentration measurement when the concentration measurement plateaus (including all Z slices) is used. The concentration measurements are pooled for each scene and summary statistics like mean, standard deviation are derived.

### Example 5. Surface Coatings to Enable Enumeration and Identification of Charged Particles

Device surface treatment was performed. Glass coverslips were stored in 1M sodium hydroxide overnight followed by an overnight wash with Hyclone High Molecular Biology Grade Water. Coverslips were air dried and plasma treated with a Harrick PCD-32G Plasma cleaner. This treatment results in a negatively charged glass surface suitable for the attraction and attachment of some viruses to the glass surfaces of the device. To create a positively charged surface, coverslips were coated with 10 μg/mL poly-L-lysine (MP Biomedicals) for 30 minutes at room temperature followed by three washes with Hyclone High Molecular Biology Grade Water. Coverslips were then left to air dry and stored at 4° C. Before assembling as above. Samples were stained with 1×SYBR Gold II and diluted in Hyclone Molecular Biology Grade Water as appropriate. Samples were then loaded into the Virometer via capillary action and incubated at room temperature protected from light for 90 minutes.

Poly-L-lysine may be substituted by any protein or functionalization molecule that can be physiosorbed or chemically conjugated to the glass surface to alter the surface charge to varying degrees (e.g. strongly negative, vs weakly positive-across an entire range depending on the molecules deposited). The glass surface can also have molecules attached to passivate and create a neutral charge if desired.

### Example 6. Method of Nano-Scale Particle Enumeration and Characterization in Free Solution

A simple device called the NanoVis in one iteration consisting of two coverslips and double-sided tape can be created on demand and loaded with as little as 5 μL of suspended sample in a manner similar to a hemocytometer. Samples are imaged on an epifluorescent microscope in one of two ways. For simple enumeration, z-stacks are taken across the sample, and custom image analysis is used to segment the resulting images and determine the count by averaging across the imaging fields. For size information, time lapse images of single visual fields are taken at 33 fps over a duration of 15 seconds. Each particle is then tracked to measure the length of its path over the given time and determine the mean square displacement. The mean square displacement is used to calculate the diffusion coefficient based on Brownian motion. Constant collisions with surrounding molecules in a fluid medium cause suspended particles to randomly fluctuate in position, resulting in Brownian motion. The smaller the particle, the more prone it is to these fluctuations. The diffusion coefficient of each particle can be used to calculate the diameter via the Stokes-Einstein equation. By applying local intensity thresholding rules on each image, it is possible to detect particles of varied sizes and fluorescence intensities, overcoming some of the limitations of traditional light scattering-based nanoparticle tracking analysis (NTA). Polydisperse EVs derived from pancreatic ductal adenocarcinoma cells (Panc1) were stained with a covalent fluorescent dye and measured in this way.

### 1. Materials and Methods

### 1.1 Device Assembly

3M Double Coated Tape 9500PC was cut into strips using a US Cutter Model SC vinyl cutter. Two strips of tape per desired sample were applied to a 24 mm×60 mm Gold Seal pre-cleaned #1 coverslip. A second coverslip was then placed on top of the tape in a way that left a lip at the edge for sample loading. Samples were loaded into the open-ended chamber that was formed between the two coverslips with tape as the spacer.

### 1.2 Sample Preparation

### 1.2.1 Fluorescent Bead Preparation

50 nm Dragon green polystyrene microspheres (Bangs Labs) were diluted in Hyclone Molecular Biology Grade water and bath sonicated for 30 seconds in a Fisher Scientific Ultrasonic Bath Cleaner. 200 nm Dragon green polystyrene microsphere were similarly diluted in Hyclone Molecular Biology Grade water and bath sonicated for 60 seconds. Following sonication, microspheres were further diluted in Molecular Biology Grade water as needed.

### 1.2.2 Cell Culture

Pancreatic cancer (PANC-1) cells were seeded in T175 culture dishes in Dulbecco's Modified Eagle Medium (DMEM, Sigma-Aldrich) supplemented with 2% FBS (Atlanta Biologicals) and maintained in a humidified chamber at 37° C. and 5% CO2 until they reached 70-80% confluence. To begin EV production, the culture dishes were twice washed with phosphate buffered saline (PBS) and serum free DMEM was added. After 24 hours, the conditioned media was collected and spun down at 2000× g for 10 minutes each to remove cellular debris and large particles (>2000 nm).

### 1.2.3 Extracellular Vesicle Isolation, Concentration, and Staining

EVs were isolated from the conditioned media via ultracentrifugation. To isolate large vesicles (LVs), the media was spun down at 12,000×g at 4° C. for 20 minutes. The media was transferred to new centrifuge tubes and the LVs were resuspended in 50 ul of PBS.

To isolate microvesicles (MVs), the media was spun down at 20,000×g at 4° C. for 60 minutes. The media was again transferred to new centrifuge tubes and the MVs were resuspended in 50 ul of PBS. To isolate small vesicles, the media was spun down at 100,000×g at 4° C. for 5 hours. The pellet was resuspended in 50 ul PBS. The leftover media was concentrated via filter centrifugation using a VivaSpin 20 centrifuge tube with a molecular weight cutoff of 20 kDa per manufacturer's instructions. All samples were stored at −80° C. for further analysis. Prior to analysis, EVs were thawed on ice and diluted in 1:25 in phosphate buffered saline (PBS). EVs were stained with 16 μM CFSE (Cell Trace CFSE Cell Proliferation Kit, Thermo Fisher Scientific) for 2 hours at 37° C. alongside an EV-free buffer control for fluorescent imaging. The stained buffer control showed no particles or aggregates.

### 1.3 Analysis

### 1.3.1 Transmission Electron Microscopy (TEM)

SiO2 wafers were first cleaned with detergent, acetone, and IPA. Passivated with 0.2M NaOH for 30 minutes. Activated with (3-aminopropyl)triethoxysilane (APTES, 5% v/v in 95% IPA, 10 min. GA (1% v/v in 1×PBS, 1 hour). EVs were covalently immobilized on top of GA for 1 hour. The remaining GA active sites were deactivated with Tris-ethanolamine (Tris-ETHA, 0.1M Tris buffer and 50 mM ethanolamine, pH 9.0, 30 minutes (I used run buffer which is TRIS-TEA-Tricine). The functionalized substrates were washed and stored with 1×PBS prior to the subsequent pre-imaging steps. Grids were coated with poly-L-lysine for 30 minutes and beads were incubated for 1 hour and stained with uranyl acetate.

### 1.3.2 Scanning Electron Microscopy (SEM)

EVs were fixed for 30 minutes in 2% glutaraldehyde in 0.1M Na cacodylate buffer then washed in 0.1M cacodylate buffer. They were then dehydrated in 25%, 50%, 75%, 95%, and 2×100% ethanol for 10 minutes each step and then dried them in the critical point dryer. EVs were sputter coated and then visualized with SEM.

### 1.3.3 NanoSight NTA Measurements

Samples were loaded into the NanoSight NS300 (Malvern Panalytical) and recorded for three 45 second videos per sample with a camera level of ranging from 13-15. The detection threshold ranged from 31-45 for samples containing 200 nm beads and 3 for samples containing only 50 nm beads. A detection threshold of 5 was used for EV samples.

### 1.3.4 Image Acquisition

Images were acquired on a Zeiss Axiovert Z1 using a 63×oil objective. For enumeration, 80 z-stacks each consisting of at least 45 slices at an interval of 0.5 μm were taken across the sample at 20% intensity and 33 ms exposure. For size characterization time lapse imaging was performed at 20% intensity using the streaming setting and recording at approximately 33 fps (exposure time=33 ms) which is comparable to the NanoSight. Time lapse durations were 15 s, and all imaging was performed at 23° C. Time lapse imaging was repeated at exposure times of 50 ms, 70 ms, and 90 ms for size calibration and correction.

### 1.3.5 Particle Counting Algorithm

Custom image processing algorithms were used for enumeration and size characterization. For enumeration, objects were tracked through the z-axis to ensure that the same object is not counted more than once. Images were adjusted for contrast and then masks for potential objects are made based on median and top-hat filtering. To obtain precise resolution of the objects, each isolated mask was locally thresholded based on the background noise around the mask. Any object that appears within a given radius of exclusion for at least 2 consecutive slices is considered one unique particle. If a particle is not detected for one slice and is detected in the next, it can still be considered the same particle. After tracking, the particles were enumerated, and the count was divided by the imaging volume to calculate the particle concentration. For size characterization, time lapses were analyzed so that the particles were tracked through the x and y-axes. Any particle that persists within the exclusion radius for at least 2 frames is considered a unique particle.

### 1.3.6 Brownian Motion Calculations

Each track was at least 15 frames amounting to a duration of ˜500 ms. Track length over a given time interval was considered to be the displacement and used to calculate the diffusion coefficient (D). The diffusion coefficient for each frame was averaged across the entire track for the relevant particle. The average diffusion coefficient of each individual particle was then used to calculate its diameter based on the Stokes-Einstein equation. The imaging volume of tracks is estimated from the average time (using the number of time-frames) each track in the bin persists through. The product of the persistence time and the diffusion constant of a track is proportional to the imaging volume. Normalizing with respect to the imaging volume gives the histogram of the track concentrations. Note that the total concentration of particles is derived from the technique for enumeration in suspension described in the previous section.

### 2. Results and Discussion

### 2.1 Conventional Epifluorescent Imaging can be Used to Count Fluorescent Nano-Scale Particles in Suspension.

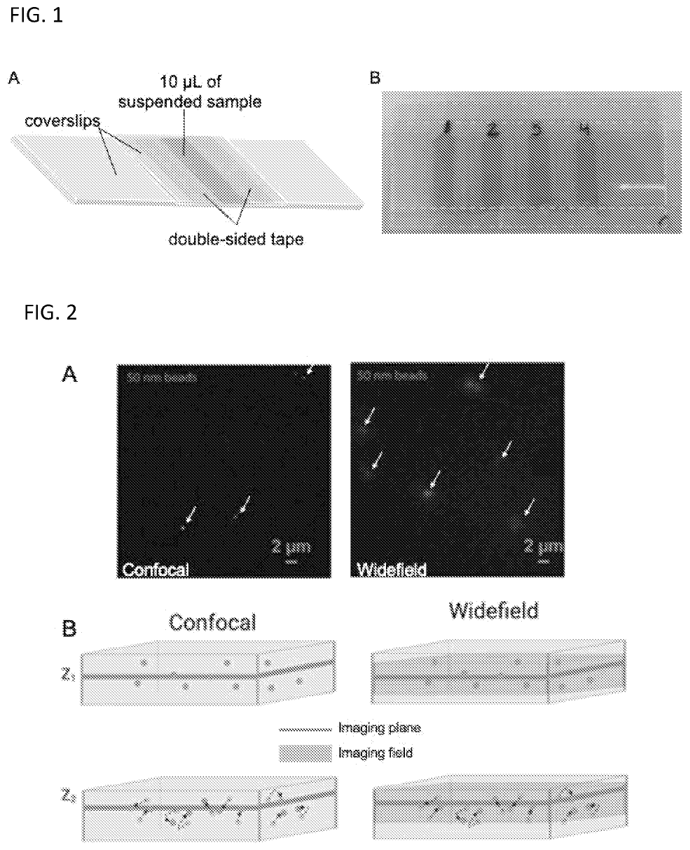

In order to image nano-scale objects and track their motion (FIG. 1A), a microfluidic chamber (NanoVis) integrated onto a coverslip for imaging a small volume of fluid with a microscope, similar in concept to a hemocytometer, was developed (FIG. 1B). The microfluidic chamber is created by cutting 120 μm-thick adhesive film (3M double coated tape 9500PC) which serves as chamber walls and a defined spacer between two glass coverslips. This creates a chamber that supports a sample volume of 10 μL for imaging and enumeration. To make the method as widely applicable as possible, nanoscale objects were imaged in free suspension, rather than relying on surface functionalization strategies to immobilize known objects to the glass surface. However, this approach requires that the effects of Brownian motion on the imaging, which are considerable at the nanoscale, are accounted for.

A method that takes advantage of the Brownian motion that these particles experience was developed in order to detect and characterize them using fluorescent microscopy. Using this system, Dragon Green 50 nm polystyrene beads (Bangs Laboratories) were imaged on a confocal microscope and a standard epifluorescent microscope in suspension using a 63×oil objective (FIG. 2A). Both microscopes resolved individual particles as smears. Confocal microscopy typically offers higher quality imaging due to its ability to focus on a single plane; however, this modality did not provide an advantage in this context as it is impossible to determine if an object that appears in a given plane is unique or if it is the same particle that was imaged in a previous plane and has traveled in space without sophisticated autocorrelation methods such as spatiotemporal image correlation spectroscopy (STICS). Given that the goal is to develop an accessible platform, a workflow that relies on widefield imaging and no complex, specialized autocorrelation procedures was chosen. By taking advantage of the thicker imaging plane in standard widefield imaging, it possible to determine if each particle is unique by tracking its movement through the Z plane (FIG. 2B).

### 2.2 Nano-Scale Particles can be Enumerated in Suspension.

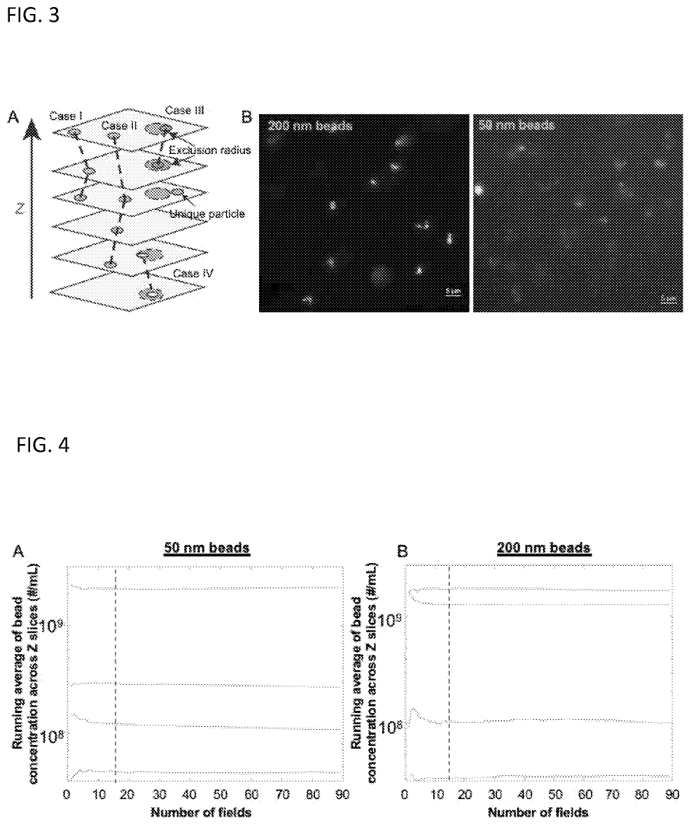

Z-stacks were taken of the beads in solution at a resolution of 500 nm per slice for a total imaging depth of 25 μm. Due to a combination of bleed-through and the constant motion of the particles, it is necessary to track each particle through the z-axis using custom image analysis algorithms (FIG. 3A). If a particle persists in the same xy coordinates across multiple slices, it is considered to be same particle (Case I). If a particle disappears for one slice and is visible in the next, it is assumed that it is the same particles that experienced a brief fluctuation out of the field of view (Case II). Furthermore, the identity of a particle is estimated on the basis of it remaining within a certain radius of its predecessor in a previous slice (Case III). The exclusion radius is determined based on the minimum size particle you wish to detect. This requires a priori knowledge of the size range that is expected. However, the algorithm allows a user to perform a verification step where they ensure that the algorithm is properly tracking the appropriate particles, which would allow the user to determine the exclusion radius that accurately captures unique particles. Tracking the particles through space and time in this manner reduces the possibility of counting the same particle more than once. This method is comparable to spatiotemporal image correlation spectroscopy (STICS), a confocal-based correlation method wherein an object is tracked across two frames and temporal persistence is calculated to determine whether a particle is unique. However, this approach requires access to a confocal microscope. To show that this method can be applied to particles of multiple sizes, Dragon Green 200 nm and 50 nm polystyrene beads were enumerated in suspension by fluorescent imaging (FIG. 3B). The number of particles was divided by the z-volume to determine the concentration.

To determine how many replicate fields of view must be taken to achieve the statistical relevance for an accurate count, z-stacks were taken of 100 fields, and a running average was taken to establish the point at which the counts began to converge towards one solution (FIG. 4). Approximately 20 fields of view were determined to be adequate for a precise count.

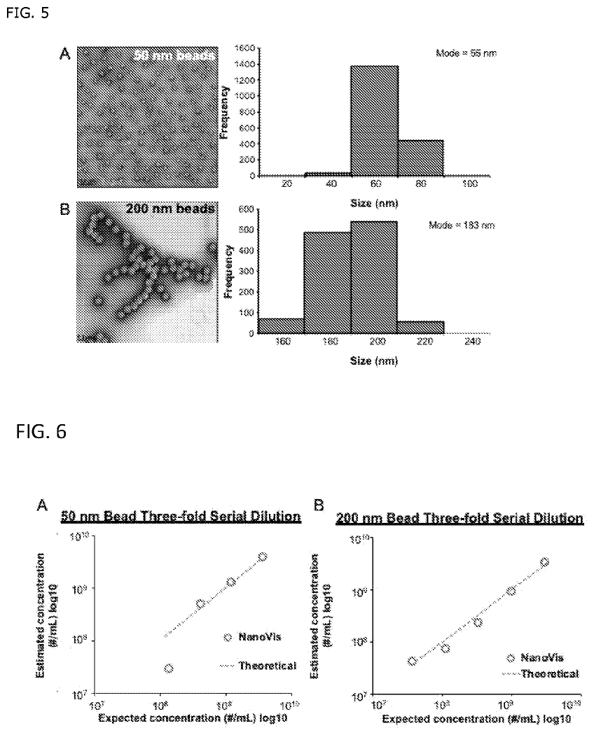

50 nm beads and 200 nm beads were imaged via TEM revealing modal diameters of 55 nm and 183 nm, respectively (FIG. 5).

The size information obtained via TEM was used to calculate the expected concentration of the beads by determining the volume of a single bead and estimating how many beads would occupy the volume reported by the manufacturer of the beads. A three-fold serial dilution was performed to ascertain the range of concentrations for which this method is accurate, revealing that counts that matched the expected concentration for a range of 10^{7.5}-10^{9.5} for 200 nm beads and 10⁸-10^{9.5} for 50 nm beads (FIG. 2.6). A deviation from the expected concentration was observed at the low concentration for the 50 nm beads. Linear best fit lines for the correlation of expected and estimated concentrations for the 50 nm beads and 200 nm beads had slopes of 1.2 and 1.1, respectively. This represents a robust, low cost method for quick and accurate enumeration of fluorescent nano-scale particles.

### 2.3 Particle Tracking can be Used to Reveal Size Distribution of Suspended Nano-Scale Particles.

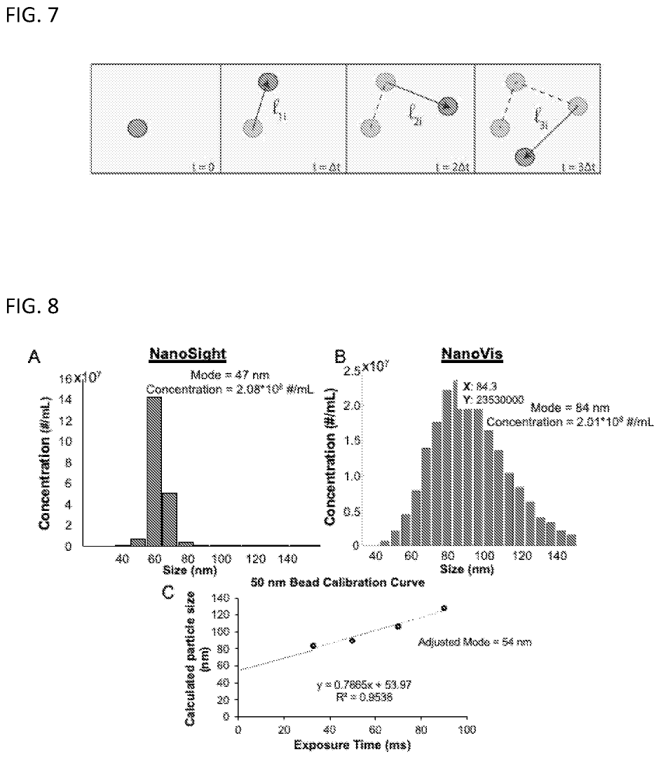

Although taking advantage of Brownian motion to image smears rather than point particles lends itself to facile enumeration, size information cannot be obtained by direct measurement. However, by taking time lapse images of smears that serve as proxies for particles, it was possible to track their motion and calculate the diffusion coefficient. Smears were imaged in a single frame every 33 ms for a total of 15 s, and their paths were measured by tracking their centroids (FIG. 7). The displacement of each smear was determined based on the distance between centroid location at one time point compared to the next. The diffusion coefficient was calculated for each Δt based on the following formula for 2D diffusion (Equation 1):

$${Δ}_{ms} = 4 D Δ t \qquad Equation 1$$

The distance traveled by a particle during the duration of the frame (Δt) is considered the mean squared displacement (Δms) and is used to calculate the diffusion coefficient (D). The calculated diffusion coefficient for each particle across all time points was averaged to determine the diffusion coefficient for that particular particle. The diffusion coefficient was then used to calculate the diameter of each particle (d) at a given temperature (T) and medium viscosity (η) with the Boltzmann constant (kb) using the Stokes-Einstein equation (Equation 2) as follows:

$$d = \frac{{k}_{b} T}{3 πη D} \qquad Equation 2$$

The custom algorithm that was used for tracking determined the identity of each smear through time based on its presence in 2 frames in a row. Since Brownian motion is random, the Stokes-Einstein equation's accuracy is derived from the measurement of large numbers of particles. Rather than measuring a large number of particles, measurements of the same particles are repeated over time and averaged to determine the overall trends. The concentration measurements are obtained by binning the tracks according to sizes and then dividing with respect to the imaging volume of tracks in each bin. NanoSight analysis revealed a modal diameter of 47 nm (FIG. 8A). The same beads were imaged on the NanoVis at an exposure time of 33 ms, resulting in a modal diameter of 84 nm (FIG. 8B). This inflated measurement is likely due to the fact that an exposure time of 33 ms is capturing a smear left by a moving particle rather than a punctate signal representing the exact location of the particle. The beads were then imaged across a range of exposure times (50 ms, 70 ms, and 90 ms) with smear size increasing with exposure time. Each exposure time condition comprised over 13,500 frames, ensuring a large sample size for the size estimates. The resulting calculated sizes were fit to a linear curve to determine the correction factor for extrapolating bead size at a theoretical exposure time of 0 ms (R²>0.95) (FIG. 8C). This yielded a particle size of 54 nm which is in close agreement with the TEM measurements (55 nm).

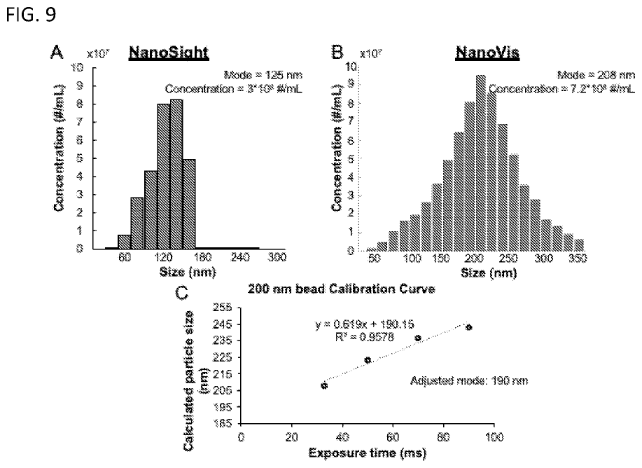

NanoSight analysis of the 200 nm beads revealed a modal diameter of 125 nm (FIG. 9A). NanoVis analysis at an exposure time of 33 ms showed a modal diameter of 208 nm (FIG. 9B) corrected to 190 nm using a linear calibration across a range of exposure times (FIG. 9C) which is within 10 nm of the measurements obtained using TEM (183 nm).

When compared to the NanoSight, the NanoVis displays broad size distributions for both the 50 nm and 200 nm particles, likely due to the noise inherent to the system. To determine how much noise originated from the uncertainty of the size calculation compared to the true variance of the particle size, an error analysis was performed to calculate the bias and precision of this platform. The mean of the NanoVis size data (μcombined) was assumed to be a combination of the mean of the actual size of the particles (μparticles) and the mean of the measurement process noise from the NanoVis (μmeasurement) (Equation 3).

$${μ}_{combined} = {μ}_{particles} + {μ}_{measurement}$$

The mean of the TEM measurements (FIG. 5) served as μparticles because the measurements were obtained by directly counting pixels from TEM images from a large number of samples (n>1000) and assumed to have negligible process noise. The TEM measurements displayed a normal distribution for both bead sizes as did the NanoVis data. A μmeasurement, or bias, of 37.45 was calculated for the 50 nm particles and 17.7 was calculated for the 200 nm particles (Table 1).

Similarly, the total variance of the NanoVis measurements (SD²_{combined}) was assumed to be a combination of the variance of the actual size of the particles as determined by TEM (SD²_{particles}) and the variance caused by measurement process noise (SD²ₘₑₐₛᵤᵣₑₘₑₙₜ) (Equation 4).

$${{SD}^{2}}_{combined} = {{SD}^{2}}_{particle} + {{SD}^{2}}_{measurement}$$

SDₘₑₐₛᵤᵣₑₘₑₙₜ represents the precision of the measurement and was calculated to be 33.6 for the 50 nm beads and 93.8 for the 200 nm beads (Table 1). In summary, the NanoVis displays greater accuracy for larger particles compared to small particles, necessitating a size calibration (FIG. 8C and FIG. 9C). High noise and low precision were observed for both bead sizes. Although the size of the particles can be estimated using the described size calibration method with high accuracy, the NanoVis may not be ideal for applications in which high precision is a priority. However, in some cases where the priority is simply determining the modal diameter this may be an acceptable tradeoff given the low cost and ease of use.

TABLE 1
Summary of bias and process noise in 50 nm and 200 nm beads
50 nm beads

| | | | |
|---|---|---|---|
| μ_{combined} | 93.0 | SD_{combined} | 34.31 |
| μ_{particles} | 55.5 | SD_{particles} | 7.1 |
| μₘₑₐₛᵤᵣₑₘₑₙₜ | 37.45 | SDₘₑₐₛᵤᵣₑₘₑₙₜ | 33.6 |

200 nm beads

| | | | |
|---|---|---|---|
| μ_{combined} | 200.0 | SD_{combined} | 94.7 |
| μ_{particles} | 182.3 | SD_{particles} | 12.6 |
| μₘₑₐₛᵤᵣₑₘₑₙₜ | 17.7 | SDₘₑₐₛᵤᵣₑₘₑₙₜ | 93.8 |

### 2.4 Polydisperse Particles can be Tracked and Characterized.

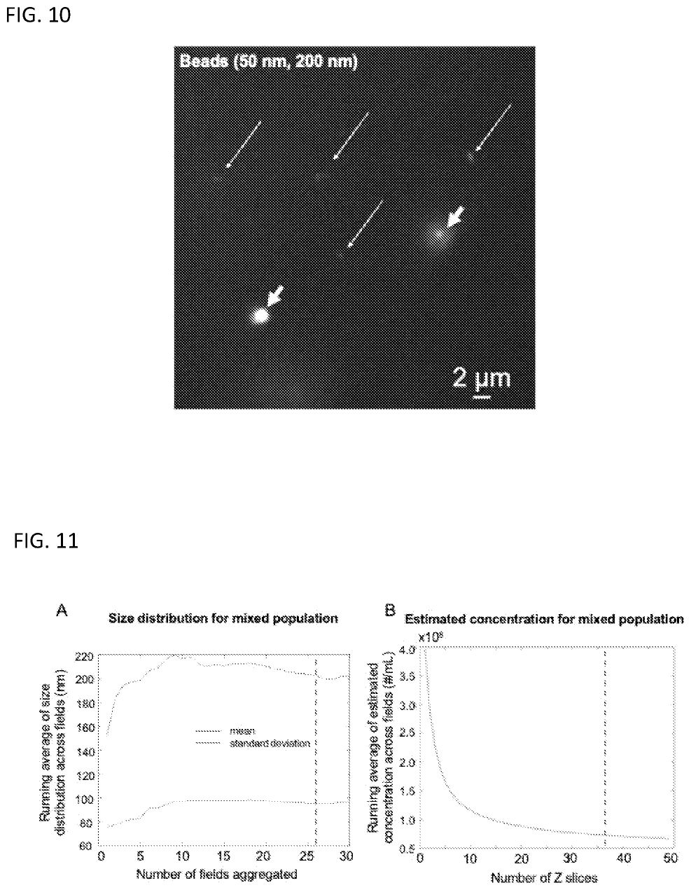

Although the enumeration and size characterization of homogenous particles in suspension is informative, it is oftentimes necessary to characterize mixtures containing particles of varying sizes. Traditional light scattering-based NTA often misses smaller particles in the presence of larger particles that effectively drown out their signal. This could result in an inability to detect contaminating particles in a supposedly homogeneous mixture or even a failure to distinguish important components of a heterogeneous mixtures, such as EVs released from cells. 200 nm and 50 nm particles were mixed into a single suspension and imaged using the NanoVis. By adjusting the thresholding of the image, both the bright 200 nm particles and the relatively dim 50 nm particles were detected (FIG. 10).

Based on the running average of the size data. it was determined that between 25-30 fields of view of time lapse data were sufficient for size characterization of this mixed population (FIG. 11A), and between 35-40 fields of view were sufficient for accurate enumeration (FIG. 11B).

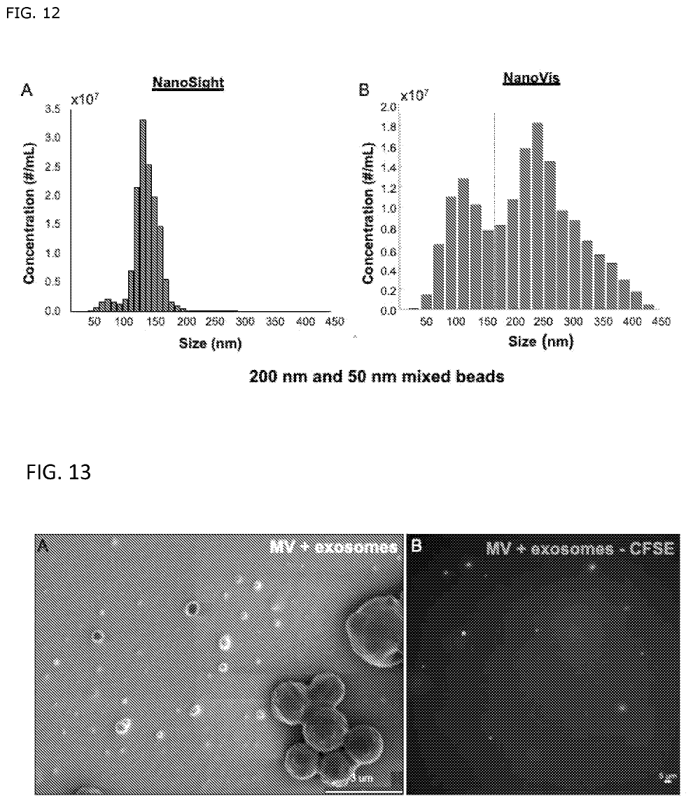

The NanoSight successfully captured a bimodal population, (FIG. 12A). However, it also undercounted the 50 nm particles, which were expected to be equal in number to the 200 nm particles. Adjustments to the NanoSight settings such as increasing the camera level and lowering the detection threshold were not sufficient to overcome its inability to detect the full population of smaller particles among the highly refractive large particles. A likely explanation is that the camera level requiring to detect the 50 nm particles resulted in the 200 nm particles emitting multiple points of scattered light, causing them to be overcounted relative to the 50 nm beads, which were largely masked by the signal from the 200 nm beads. Although the modal diameters for each peak were higher than expected, the NanoVis revealed the expected bimodal population of beads (FIG. 12B). Both methods displayed a compromise in accuracy in the mixed sample with the NanoSight underestimating the size of the 200 nm beads and the NanoVis overestimating the size. However, the NanoVis did display the ability to resolve two populations in a mixture at the correct ratio. Proper size corrections would increase the accuracy of the NanoVis for mixed samples. 2.5 Extracellular Vesicles (EVs) can be Enumerated and Characterized Using the NanoVis

A mixture of suspended Panc1 microvesicles (MVs) and exosomes was imaged via SEM, revealing a heterogeneous population of EVs (FIG. 13A). The fluorescence-based method was applied to EVs stained with the cell permeable covalent dye carboxyfluorescein succinimidyl ester (CFSE), showing an ability to resolve individual extracellular particles (FIG. 13B).

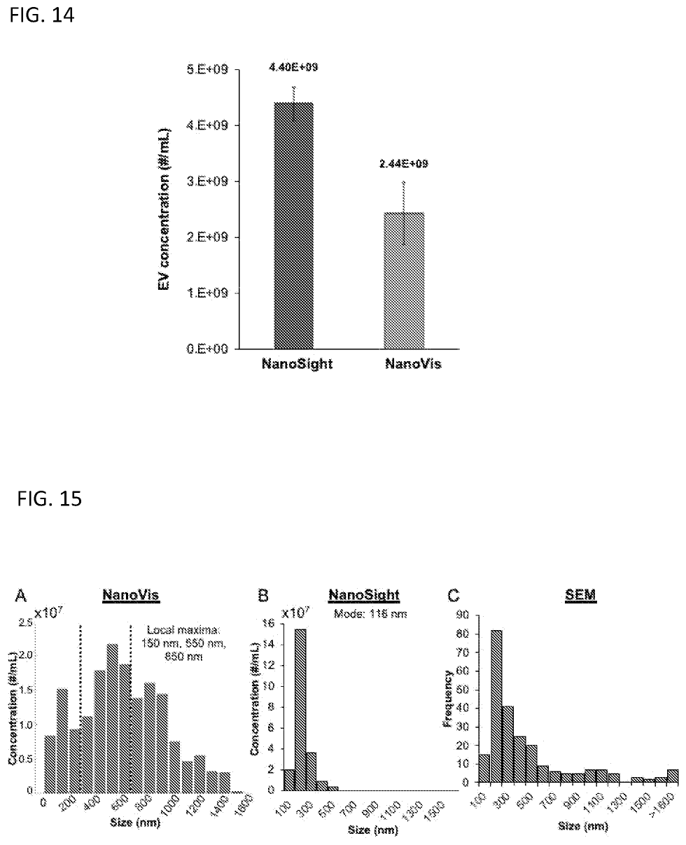

The particles were enumerated on the NanoVis and on the NanoSight revealing concentrations within an order of magnitude of one another, with the NanoVis concentration being roughly half of what was observed on the NanoSight (FIG. 14). This is not surprising as the NanoSight has been found to overestimate EV concentration relative to other methods. Additionally, the NanoSight can detect non-specific particles or cell debris via light scattering, whereas the NanoVis only detects particles that stain positively for CFSE.

Size analysis on the NanoVis revealed a multimodal population spanning from the nano-scale to the micro-scale (FIG. 15A), which is in agreement with the sizes measured on the SEM, whereas the NanoSight only detected the nano-scale particles (FIG. 15B), indicating that the NanoVis better captures the heterogeneity inherent in biological samples such as EVs (FIG. 15C). Current methods for enumerating an characterizing EVs are heavily prone to missing smaller particles, such as exosomes. In fact, it is possible that the NanoSight counted some of the larger particles as multiple smaller particles because they scattered multiple points of light, contributing to the tendency of the NanoSight to overcount EVs. Since freshly isolated EVs are an inherently heterogeneous population consisting of exosomes and other larger particles, it is important to accurately characterize the full range of particle sizes.

### 3. Discussion and Future Directions

The ability to resolve individual suspended fluorescent nano-scale particles on a widefield microscope using a simple device consisting of two cover slips and double-sided tape was demonstrated. Particles were enumerated, and concentration values that closely matched the theoretical values based on the size of the particles as determined through TEM were obtained. The error intrinsic to this system was taken advantage of to accurately calculate the size of monodisperse populations by applying a correction factor. This method allows for the detection of multimodal populations without the signal being drowned out by the larger population as is common in other NTA-based platforms. The NanoVis was also able to enumerate and characterize the size distribution of a mixed MV and exosome population by capturing both nano- and micro-scale particles, whereas the NanoSight preferentially captured only the nano-scale objects.

Further studies should focus on determining the size resolution that the NanoVis is capable of and how it compares to other methods such as the NanoSight. The NanoVis can resolve separate populations of 50 nm and 200 nm particles, and the next step would be to look at three known populations as well as known populations that are closer in size. Future directions include performing adjustments so that size corrections are applicable to samples with mixed populations. The results obtained using the NanoVis were compared to EM and the NanoSight, but it would also be beneficial to compare results from the epifluorescent microscope to confocal images that were processed using autocorrelation to further validate the accuracy of the custom image processing algorithm.

Imaging settings were optimized for the epifluorescent microscope that was used for experiments. However, anyone who wishes to adapt this method in their own lab needs to confirm the imaging settings are ideal for their microscope given that light intensity can vary with the type and age of the light source. Prior to applying this method to a biological sample, a user should validate the NanoVis with fluorescent beads of known size to eliminate variability based on microscope settings or sample fluorescence intensity. For example in the case of widefield microscopy, LED light sources and halogen or arc lamps have different spectral properties, as the former emits a single wavelength at a wide bandwidth, whereas the latter relies on optical filters to transmit a narrow wavelength of light from a broad emission. Furthermore, most microscopes allow the user to adjust the illumination intensity as a function of the percentage of the maximum possible intensity. However, the nominal value of the maximum intensity of the light source is rarely reported and also diminishes with time. As such, it is necessary to determine the appropriate imaging settings based on the light source of the microscope being used. The exposure time settings for size analysis should not vary from this protocol as they are important for diffusion calculations. Once the appropriate imaging settings have been chosen, the user must perform a size calibration across a range of exposure times to calculate the correct size (as seen in FIG. 2.8C and FIG. 2.9C). Nano-scale particles of known size, such as commercially available polystyrene beads, can serve as validation that the linear calibration is producing accurate size predictions. Doing so is important because different microscope settings may result in different smear sizes that require different correction factors. Once the calibration has been shown to work on particles of a known size, the user can apply this method to particles of unknown size.

Furthermore, it is important to ensure that sample processing is appropriate for fluorescent imaging in suspension. The sample processing steps described herein for polystyrene beads and Panc1 EVs are unlikely to be appropriate for nanoparticles made of different materials or for EVs that are being stained using alternative dyes or that require other types of downstream processing. Care must be taken to avoid any clumping in the sample as it will compromise both count and size information. In the absence of access to EM, clumping can be detected by applying a range of methods for mixing (such as a range of settings for probe sonication, bath sonication, vortexing, or others as deemed appropriate) and comparing the counts obtained with each. If the counts differ across mixing conditions, it is likely that there is clumping or that the particles are being destroyed and leaving debris. Size analysis can be performed to determine if either is the case with the assumption that clumping will cause the size estimate to be too large, and debris will result in a population of particles that are smaller than expected. Additionally, it is important to confirm that any stains that are used on the sample do not alter the characteristics of the particles. For example, lipophilic dyes such as PKH are known to alter EV size. Dye-induced changes in EV characteristics can be detected using SEM or by comparing light scattering-based NanoSight measurements for stained and unstained samples to see if the size or number of particles change. In the absence of these methods, one could perform a screen of different dyes to determine if size or quantity are altered with the caveat that it is difficult to ascertain which measurement is “correct.” It is also important to analyze blank sample-free controls containing the dye of interest because some dyes form fluorescent aggregates that are easily detected and can cause false positives.

In addition, it is important to determine if a polydisperse sample is expected to display uniform fluorescence intensity independent of size or if the fluorescence intensity of the particle correlates with size. Differences in fluorescence intensity have implications for both imaging settings and thresholding settings during downstream image analysis. If sample processing allows, the user could analyze samples containing only particles that are expected to have a smaller size and compare them to samples only containing particles of a larger size to see if their baseline fluorescence intensities differ. If this is not an option, the user could adjust the thresholding on their images to determine if dimmer particles appear. In this case, a blank sample-free control would be necessary to confirm that any dim particles that are observed are not artifacts from the medium. Size correction was performed for nano-scale objects, but it is less likely to be necessary for micro-scale objects because they experience less Brownian motion and would therefore be expected to display more consistent movement patterns with less variability across exposure times. However, confirming this in micro-scale objects of a known size would be beneficial.

Ideally, initial results will be ground-truthed against an established method prior to adopting the NanoVis in a new lab. Although democratization is the goal of this platform, a new user would benefit from an upfront investment in analyzing their sample using a more costly but established method to be confident in the results obtained using the NanoVis. For example, a side-by-side analysis comparing measurements performed using the NanoVis on an epifluorescent microscope and processed with the algorithm described herein to measurements obtained from confocal images processed using autocorrelation would serve as a good validation that the particles are being accurately detected and tracked and that all size ranges are properly accounted for. Higher concentrations in the confocal-imaged sample could indicate that the epifluorescent microscope is not adequately detecting every particle. The size distribution reported following an established autocorrelation-based analysis would serve as a reasonable benchmark for the NanoVis since the methods used to determine size are comparable. If the distribution of the autocorrelation data is narrower than the distribution of the NanoVis data, then it is likely to detect peaks that are lost to the noise in the NanoVis data. If no additional peaks are detected, then NanoVis data can be assumed to be accurate with regard to how many modes exist in the sample. If the data do not agree, then it may be necessary to adjust parameters within the image processing algorithm to ensure that each particle is being properly tracked. Once the user is confident in the quality of their sample and that the NanoVis is providing acceptable results based on their needs (as determined by validating with known samples and comparing to established methods), they can incorporate this platform into their routine workflow.

### Example 7. Method for Direct Enumeration of Cultured and Environmental Viruses

Here a simple low-cost method for on-demand virus enumeration is presented in which suspended SYBR-stained viral isolates can be seeded into a device consisting of two coverslips separated by double-sided tape and left to naturally adhere to treated glass. As little as 5 μL of sample is sufficient for this method, leading to less wasted sample and less wasted time in determining the proper dilution and allowing for greater throughput. Coverslips were either plasma-treated to create a negative charge or treated with poly-L-lysine (PLL) to create a positive charge, depending on the projected isoelectric point of the sample. Following adhesion, the surface of the coverslip was imaged using epifluorescence, and the images were processed using a custom counting algorithm, eliminating the subjectivity and user variability inherent to most common counting methods. Cultured T7 bacteriophage was used as a proof-of-concept to validate the accuracy of this novel method against more established methods (specifically digital PCR (dPCR), NanoSight, and Anodisc) because it is well-characterized and can be enumerated via methods that are limited to known viruses. T7 is a dipole with a slight net negative charge and was therefore imaged in a positively-charged PLL-coated device. Environmental aquatic isolates from a range of bodies of water were also counted using the Anodisc and a negatively charged device called a Virometer in this application (pH and salinity of the media result in a positive isoelectric point for these samples). Final counts between the two methods were in agreement, indicating that this method is a viable substitute for the Anodisc.

### 1. Materials and Methods

### 1.1 Virus Isolation and Processing

### 1.1.1 Preparation of Viral Lysate Standard

An overnight culture of strain Escherichia coli B (Carolina Biological Supply, Cat #124300) was grown in Luria-Bertani (LB) broth until turbid and diluted to reach an optical density of 600 nm (OD600). Culture plates were prepared using the top agar layer method. Lyophilized and vacuum-dried stock of Escherichia phage T7 filter paper (DSM No. 4623, Leibniz Institute, Braunschweig, Germany) was placed on the top agar once solidified and 100 μL of working Phage Buffer was added to allow for diffusion of phage. Plates were incubated at 37° C. for 8 hours to allow plaques to form. All plaques formed after this incubation were harvested and resuspended in 1 mL PBC. The resuspension was centrifuged at 15,000 rcf for 10 minutes and filtered through a Whatman ANOTOP™ syringe top filter with 0.2 μm pore size (GE Healthcare UK Limited, Buckinghamshire, United Kingdom) to create a preliminary T7 phage lysate. The preliminary lysate was quadrant streaked on additional E. coli B plates and plaque purification by isolation from a single plaque was performed to create a pure liquid culture of Escherichia phage T7. This pure liquid culture was then added to E. coli B culture with molten top agar (50° C.), poured onto plates and allowed to incubate for 8 hours. Plates were then flooded with 5 mL PBC and placed on a shaker for 1 hour at room temperature to harvest phage from the media. The working T7 lysate is used for all downstream applications. Virometer and Anodisc counts were performed to estimate the concentration and calculate a working dilution for digital PCR applications.

### 1.1.2 Environmental Sample Collection

Environmental samples represent a gradient of aquatic environmental conditions. Samples labeled “BATS” come from a research cruise collection at the Bermuda Atlantic Time Series station (31°50″N 64° 10″W) in August 2019 and represent a marine environment. Raw samples were not processed through filtration. Viral Concentrate (VC) samples were filtered through ISOPORE™ 3 μm (TSTP14250 EMD, Millipore Sigma, Burlington, MA) and 0.22 μm (GPWP14250, Millipore) 142-mm filters. The filtrate was concentrated using tangential flow filtration (TFF, Pellicon 2 100 kD Lot no. C9EA39951-0033 and PelliconXL 50 kD Lot no. C9EA39921-0024, Millipore). Replicate two mL samples for counts were collected from the TFF retentate, fixed with 0.02 μm filtered formaldehyde (final concentration of 1% formalin), snap frozen in liquid nitrogen, and stored at −80° C. following standard protocols. Collection depths from the BATS study were chosen to correlate environmental metadata with abundance and metagenetic data that is not pertinent to this study, but will be described in later research. Additional sample collections were performed across three days in July 2020. The “CD Canal” represents a brackish water environment from the Chesapeake-Delaware Canal in Chesapeake City, MD (39°31′46.5″N 75° 48′42.0″W). The samples “Creek” from White Clay Creek state park in Pike Creek, DE (39°42′40.2″N 75° 41′41.0″W) and “Pond” from the retention pond behind laboratory buildings at 15 Innovation Way, Newark, DE (39°40′37.0″N 75°44′03.7″W) represent freshwater environments. Fifty mL volumes of brackish and freshwater samples were filtered using serial 3 μm and 0.22 μm syringe-top filters (GE Healthcare UK Limited) to separate viruses from larger organisms. Replicate 2 mL filtrate samples were then fixed, snap frozen and stored at −80° C. for preservation as described above. Samples were processed within two weeks of the collection date.

### 1.2 Anodisc Preparations

Viral abundance of “Anodisc” samples was determined using epifluorescent microscopy of three technical replicates (a single replicate was used to count BATS samples given limited sample volume). Whatman Anodisc™ 0.02 μm 13 mm membrane filters (GE Healthcare UK Limited) were used for all analyses heretofore labelled “Anodisc”. Viruses were stained for visualization using SYBR™ Gold (Invitrogen, Waltham, MA) and enumerated using the SYBR™ protocol of Noble & Fuhrman (1998) with minor modifications. Briefly, 1-500 UL of sample (volume dependent on sample concentration) was vacuum filtered onto a 13 mm diameter 0.02 μm membrane filter. Filters were stained 400 μL of 2.5×SYBR™ Gold in the dark for 2 minutes and vacuum filtered for a second time. Anodiscs were then air dried and mounted onto a glass slide with 12 μL of Antifade (0.1% p-phenylenediamine antifade solution in 50/50 glycerol/PBS). All Anodisc samples were imaged and counted on the same day as prepared to negate any photobleaching effects, and a total number of virus-like particles (VLPs) per mL were back-calculated for the initial sample.

### 1.3 Anodisc Imaging

For all Anodisc microscopy counts, viral abundance was determined using the Olympus BX61 epifluorescent system with a DAPI filter set under 100× oil objective. ImageJ (version 1.53h, Feb. 4, 2021) software was used with the Micro-Manager plugin (version 2.0.0 gamma) for imaging and counting purposes. Between 10-20 still-frame images were collected from each filter to get an average VLP count per field of view. VLP concentration per mL was calculated based on the field of view area and volume filtered onto the Anodisc. Three technical replicates were carried out for each sample examined and averaged for a mean concentration of viruses in the total sample. Descriptive statistics including standard deviation (SD), median count (MED), and standard error of the mean (SEM) were performed on all technical replicates for each sample.

### 1.4 Virometer Preparations

24 mm×60 mm Gold Seal pre-cleaned #1 coverslips were stored in 1M sodium hydroxide overnight followed by an overnight wash with Hyclone High Molecular Biology Grade Water. Coverslips were air dried and plasma treated with a Harrick PCD-32G Plasma cleaner (800 mTorr, high, 60 s). If desired, coverslips were coated with 10 μg/mL poly-L-lysine (MP Biomedicals) for 30 minutes at room temperature followed by three washes with Hyclone High Molecular Biology Grade Water. Coverslips were then left to air dry and stored at 4° C.

For cultured T7 samples, 3M Microfluidic Diagnostic Tape 9965 Double Sided White tape (84 μm thick) was cut into strips using a scalpel. Two strips of tape per desired sample were applied to a PLL-treated 24 mm×60 mm Gold Seal pre-cleaned #1 coverslip. A second PLL-treated coverslip was then placed on top of the tape in a way that left a lip at the edge for sample loading.

For environmental isolates, 3M Double Coated Tape 9500PC (120 μm thick) was cut into strips using a US Cutter Model SC vinyl cutter. Two strips of tape per desired sample were applied to a plasma-treated 24 mm×60 mm Gold Seal pre-cleaned #1 coverslip. A second coverslip was then placed on top of the tape in a way that left a lip at the edge for sample loading. Samples were loaded into the open-ended chamber that was formed between the two coverslips with tape as the spacer.

Samples were stained with 1×SYBR Gold II and diluted in Hyclone Molecular Biology Grade Water as appropriate. Samples were then loaded into the Virometer via capillary action and incubated at room temperature protected from light for 90 minutes.

### 1.5 Virometer Image Acquisition

Images were acquired on a Zeiss Axiovert Z1 with Flash 4.0 camera and CoolLED illumination using a 63× oil objective at 10% intensity and 100 ms exposure time. 20-40 fields of view were taken per sample. Counts were obtained using a custom MATLAB image processing algorithm developed by Saurabh Modi. Counts for each field of view were converted to concentration by dividing by the volume of occupied between the two slides within a single field of view (area of FOV*tape thickness). In the case of the T7 samples which were adhered to two surfaces, the counts obtained on each surface were added up and divided by the same volume. The device displayed an operating range of approximately 10⁶-10⁸ VLP/mL which is equal to or larger than the operating range of competing methods such as Anodisc, NTA, flow cytometry, and PCR.

### 1.6 3D Digital Polymerase Chain Reaction

Absolute quantification of the working T7 lysate was performed by Deborah Stabley at the Nemours Biomolecular Core (Alfred I. Dupont hospital for children, Wilmington, DE) and carried out using the ThermoFisher QuantStudio 3D Digital PCR (dPCR) system (Thermo Fisher Scientific, Waltham, MA) following the manufacturer's protocol. Primers and probes were designed to target gene 1 (T7 RNA polymerase), a single copy gene that allowed for accurate quantification of viral particles. Primers sequences were BLAST against the E. coli B genome assembly on NCBI to ensure that secondary amplification would be avoided in the case that host DNA were to be present in the reaction. Primers were as follows: T7FWD 5′-GTT CAG GAC ATC TAC GGG ATT G-3′ and T7REV 5′-TCT CAT CGG TCA CGG TAA CTA-3′ (Integrated DNA Technologies INC, Coralville, IA). The fluorescent probe was designed with a 5′ 6FAM and two quenchers:/56-FAM/AC AAG CAG A/ZEN/C GCA ATC AAT GGG ACC GA/3IABKFQ/(Integrated DNA Technologies INC).

### 1.7 NanoSight Measurements

Samples were loaded into the NanoSight NS300 (Malvern Panalytical) and recorded for three 45 second videos per sample with a camera level of 15. The detection threshold ranged from 3-4.

### 2. Results and Discussion

### 2.1 A Novel Method for Imaging Fluorescently Stained Viruses

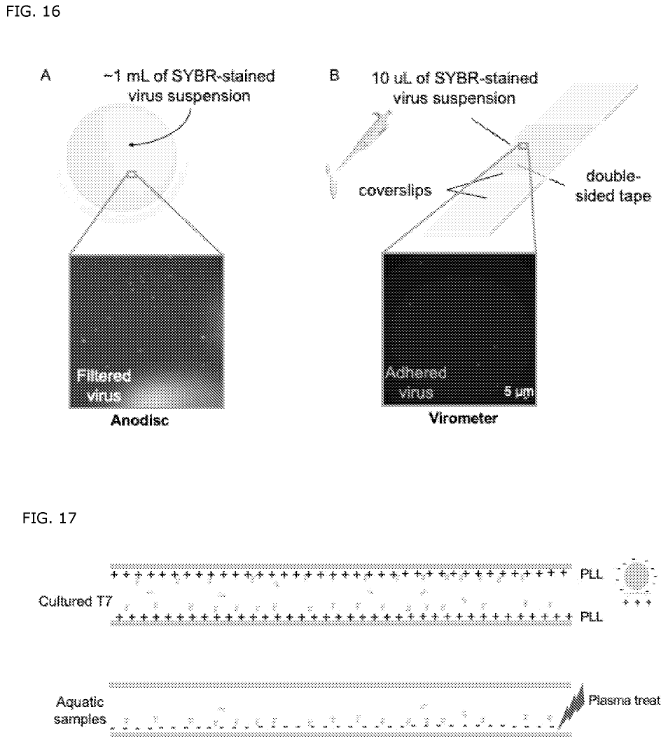

The Anodisc is considered the gold standard for virus enumeration in cases where functional assays or PCR are impractical such as when working with unknown viruses that have been isolated from the environment. These samples are often precious, especially given that researchers typically aim to perform gene sequencing in parallel with obtaining counts. The Anodisc is a ceramic filter that “catches” fluorescently-stained viruses on its surface to be imaged on an epifluorescent microscope (FIG. 16A). An alternative called the Virometer that comprises two coverslips held together with double-sided tape that can be loaded with stained virus sample similar to a hemocytometer was developed (FIG. 16B). The coverslips are functionalized to promote virus adhesion to the surface for 2D imaging. Not only does this method use less sample compared to the Anodisc, but it also requires less preparation time from the user. Individual virus-like particles (VLPs) on the surface of the Virometer were resolved.

### 2.2 Surface Treatments Allow Viruses to Stick to the Surface of a Cover Slip.

Viruses display a variety of isoelectric points (IEP) based on the composition of their capsids. As such it was necessary to develop more than one strategy to promote virus adhesion to a glass surface (FIG. 17). Poly-L-lysine (PLL) is a cationic peptide that is routinely used to functionalize surfaces to facilitate the adhesion of mammalian cells, which typically display a net negative charge. Plasma-treatment is commonly employed in the field of microfluidics to render surfaces hydrophilic and negatively charged. T7 is a podovirus with a positively charged head and a negatively charged tail. This amounts to a weak net negative charge because the head is larger than the tail.

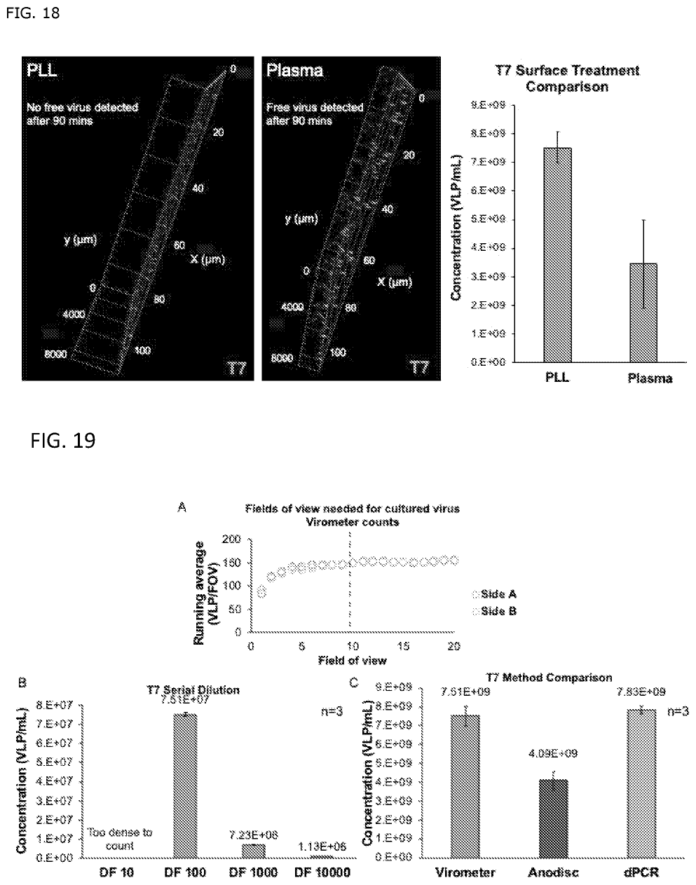

T7's adhesive properties on a positively charged PLL-treated surface and a negatively-charged plasma-treated surface were compared. Although T7 displayed the ability to adhere to both surfaces, the virus consistently showed greater adhesion to the positively-charged PLL (FIG. 18). The T7 unreliably adhered to the plasma-treated surface as evidenced by the free virus that was detected in the bulk, resulting in lower concentration counts with high variability. The T7 on the PLL-treated surface showed no virus in the bulk and higher concentration values, indicating that most or all of the virus was being captured.

### 2.3 the Virometer can be Used to Enumerate Cultured Viruses.

Due to the relatively weak charge of the T7, both sides of the Virometer had to be treated with PLL, and tape with a thickness of 84 μm was used to minimize the distance between the virus and the charged coverslip. Both sides of the Virometer were counted separately (denoted as Side A and Side B), and both sides yielded comparable VLP counts (FIG. 19A), which was expected due to the fact that gravitational forces are negligible at this length scale. The running average of each side across multiple fields of view was calculated to determine how many fields of view must be imaged in order for the VLP counts to stabilize. Approximately 10 fields of view were determined to be sufficient for an accurate statistical count. T7 was counted on the Virometer across a range of ten-fold dilutions (FIG. 19B). Concentrations greater than 10⁸ VLP/mL were too dense too count as it was impossible to resolve individual particles and because there was not enough space on the treated glass for every VLP to adhere. The Virometer displayed an operating range of approximately 10⁶-10⁸ VLP/mL which is equal to or larger than the operating range of competing methods such as Anodisc, NTA, flow cytometry, and PCR. VLP counts scaled as expected according to dilution factor, illustrating the precision of this platform. In order to evaluate the accuracy of the platform, Virometer counts were compared to the Anodisc and dPCR with dPCR serving as the benchmark (FIG. 19C). Virometer counts closely matched those obtained using dPCR, whereas the Anodisc appeared to be undercounting relative to these methods. One possible explanation could be the “meniscus effect” in which larger numbers of VLPs aggregate towards the outside edges of the Anodisc, causing the concentration in the center to be artificially low. This finding is in agreement with what was observed by Kaletta et al when they compared T7 Anodisc counts to qPCR.

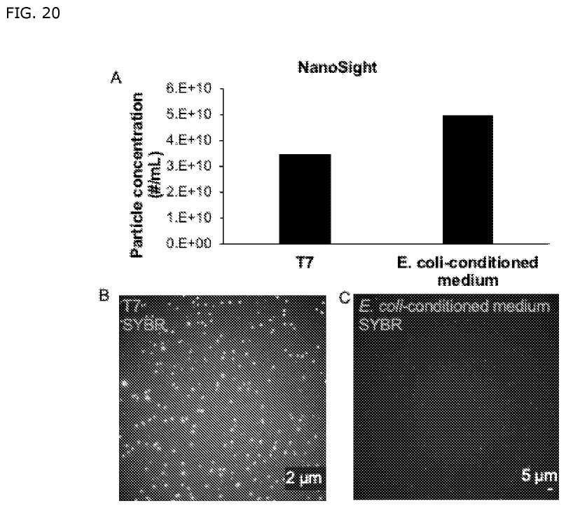

T7 was also analyzed on a NanoSight NS300, a common nanoparticle tracking analysis (NTA) instrument that relies on scattered light (FIG. 20A). NanoSight counts were 3.5*10⁹ particles/mL, almost five times as high as those obtained with the Virometer and dPCR. To test whether this was due to other factors in the media, virus-free E. coli-conditioned media was processed to determine if the NanoSight picked up any debris. Particle counts in the E. coli-conditioned media were even higher than the T7 sample, indicating that nano-scale debris left by E. coli could be contaminating the virus sample.

Comparison of images of T7 and E. coli-conditioned medium on the Virometer revealed that the virus-free samples contained particles that stained positive for SYBR (FIG. 20BC). It is possible that these particle are a type of extracellular vesicle known as outer membrane vesicles (OMVs), which are secreted by gram-negative bacteria including E. coli. These particles are faint compared to the T7 and are easily filtered out with proper thresholding during image processing. However, these findings indicate that the NanoSight may not adequately distinguish VLPs from other nano-scale particles such as extracellular vesicles.

### 2.4 the Virometer can be Used to Enumerate Environmentally Isolated Viruses.

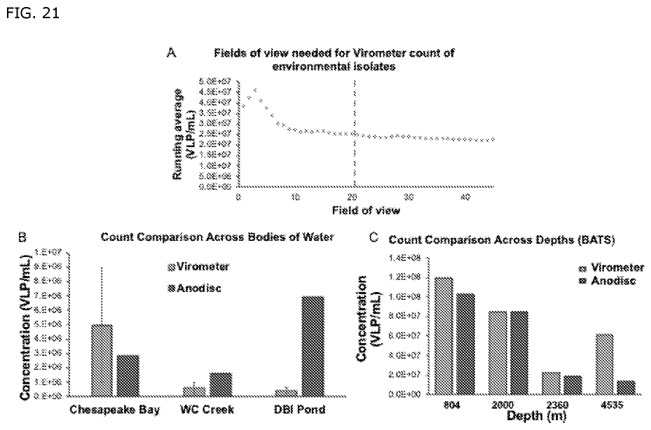

The performance of the Virometer was then evaluated using environmentally isolated aquatic viruses. The adhesion properties of aquatic viruses fluctuate based on pH, temperature, and the salinity of the medium. The aquatic samples adhered to the negatively-charged plasma-treated glass (FIG. 17). Treating only one coverslip was sufficient for attracting the viruses, as these samples empirically displayed a stronger propensity for adhesion than the cultured T7. Since environmental isolates routinely display greater variation compared to cultured samples, running averages of VLP counts were calculated across multiple fields of view on the Virometer (FIG. 21A). Approximately 15 to 20 fields of view were determined to be appropriate for a statistical count. Virometer and Anodisc counts were compared for samples isolated across multiple bodies of water (FIG. 21B). Technical replicates were obtained for the Virometer but not the Anodisc due to limited sample volume. Unsurprisingly, the environmental samples displayed greater variability compared to cultured samples in the Virometer. It is unknown if the Anodisc would have displayed similar variability. The counts for the DBI Pond showed the greatest discrepancy between the Anodisc and the Virometer. A possible explanation is the fact that viruses sometimes display a tendency towards clumping during storage, especially at low temperatures. The clumps observed by Lanni et al averaged around 10 VLPs per clump, which could ultimately translate to a ten-fold reduction in count. Not all viruses display a tendency to clump, but it is possible that the population that resides in this body of water is more prone to this behavior at low temperatures compared to those found in other bodies of water. Given that the Anodisc comprises the contents of the entire filtrate whereas the Virometer contains discrete samples that were removed from a larger sample pool, it is possible that there were aggregates of virus that were not transferred into the Virometer via pipette and therefore not accounted for. Whereas the Anodisc will reliably capture the contents of the entire sample. However, it is encouraging that the counts were within an order of magnitude of one another given that different brands of filters can often yield results that vary by up to an order of magnitude. Virometer and Anodisc counts for viral concentrates obtained across multiple depths in the Bermuda Atlantic Time-series Study (BATS) were compared (FIG. 21C). Single replicates were counted for each sample due to low sample volume. VLP concentrations were comparable between the methods across depths. Surprisingly, the Atlantic aquatic samples were more consistent between the Virometer and the Anodisc than the cultured T7. It is possible that the meniscus effect (described previously) was less powerful in these samples due to their lower concentration. These data demonstrate that the Virometer is a viable alternative to the Anodisc as it yields comparable counting data while requiring less time and fewer resources.

### 2.5 the Virometer is a Cost-Effective Alternative to the Anodisc.

Given the goal of creating democratizable platforms, the resource and time costs of the Virometer compared to the Anodisc were evaluated (Table 2). The Virometer uses a maximum sample volume of 10 μL, which translates to 0.1 μL to 10 μL depending on whether the sample is diluted beforehand. The Anodisc requires 500-1000 μL of sample, translating to 1-1000 μL depending on dilution. Sample volume becomes an important factor when considering precious environmental samples, especially if the goal is to have sample remaining for gene sequencing. Furthermore, given that the expected concentration of environmental samples is often unknown, it can be impossible to predict the appropriate dilution factor to be used prior to loading. As such, the Virometer is more forgiving in situations where a researcher must screen multiple dilution factors without wasting sample. This is similarly manifested in the person-hours required to prepare the samples. The Anodisc requires approximately one person-hour followed by one incubation-hour for a single sample. Multiple samples can be prepared in parallel depending on the availability of duplicate equipment. In contrast, preparing the Virometer requires approximately 15 minutes of in-person work followed by a 90 minute incubation. However, it is possible to quickly screen the Virometer on the microscope prior to virus adhesion to determine if the chosen dilution factor is appropriate before moving on to the incubation time, allowing the user to be more efficient with their time. Finally, the Virometer is less expensive than the Anodisc on a per sample basis. A single Anodisc costs $13 (full cost of $13.53 per sample) and serves as a single replicate, whereas a Virometer that houses up to six replicates can be manufactured for a total of $0.52 without PLL and $0.58 with PLL.

TABLE 2
Comparison of resource and time cost for Anodisc and Virometer

| | Anodisc | Virometer |
|---|---|---|
| Sample volume | 1-1000 μL | 0.1-10 μL |
| Sample preparation | 1 person-hour | 0.25 person-hour |
| time | 1 incubation-hour | 1.5 incubation-hour |
| Monetary cost | $13 - Anodisc | $0.02 - double-sided tape |
| | $0.28 microscope slide | $0.50 - two coverslips |
| | $0.25 - coverslip | $0.06 - PLL |
| | $13.53/sample total | $0.52-$0.58/sample total |

### 3. Discussion and Future Directions

The Virometer has been demonstrated as a viable alternative to both the Anodisc and the NanoSight by comparing counts of a well-characterized cultured virus (T7). Virometer counts were strongly correlated with dPCR counts and scaled appropriately in diluted samples. Altering the surface chemistry of the device allows for analysis of a wider range of samples of varying charge. This platform was successfully implemented in the context of aquatic isolates, which are notoriously more difficult to image compared to cultured samples. Although the Virometer counts for environmental isolates did not display the same precision obtained in the cultured samples, the counts were consistently within an order of magnitude of the Anodisc counts. The Virometer has the benefit of lower financial and time costs. It is also more straightforward to run replicates and to alter dilutions through the course of an experiment. As such, the Virometer has the potential to be a valuable tool in the viral ecology community.

The ideal parameters for imaging T7 were determined, but it would be necessary for researchers planning on adopting the Virometer within their own lab to validate the tool for their own virus system, whether it be cultured or environmentally isolated. Given the difficulty in resolving certain virus populations, especially in environmental isolates, it is helpful to perform validation using a well-characterized sample such as T7 to confirm appropriate imaging settings. T7 represents a sample that is easy to resolve on the Virometer and that can serve as a control to confirm that any failure to resolve viruses is not due to inappropriate imaging or analysis procedures. Furthermore, it is possible that an unknown sample may be too dilute to image. By confirming that VLPs for a known sample can be detected in the Virometer, it becomes easier to eliminate imaging settings as a source of error and to consider sources of error specific to the sample. All cultured and isolated Virometer samples described herein were imaged using the same light intensity and exposure time settings. However, it is necessary to determine the appropriate imaging settings for each individual microscope due to discrepancies in factors such as microscope light source intensity.

It is also important to confirm that all of the VLPs in a sample are adhering to the surface of the Virometer by taking z stacks and checking for floating virus in the bulk. There are a number of factors that could influence virus adherence. The isoelectric point (IEP) of a virus determines how it will react to extrinsic factors such as pH, temperature, and salinity of the media. A virus residing in a medium with a pH below its IEP would be expected to adhere to a negative surface. For example, T7 has a predicted IEP of 6.98 and would be expected to weakly adhere to a positive surface when diluted in a basic medium. Since IEPs vary widely among viruses, it is important to assess the adhesion properties of the sample in the specific medium that it will be stored and diluted in. If a sample is not adhering satisfactorily as determined by a lack of VLPs in the bulk, the user may consider diluting in a different medium or altering the conditions of the surface treatment. In the case of a positively-charged device, the PLL concentration can be increased to create more potential binding sites for the virus. A sample incubation time of 90 minutes in the Virometer was sufficient for the samples analyzed herein, but it is possible that some viruses could benefit from longer incubation times if care is taken to prevent evaporation of the sample. Depending on the strength of the sample's charge, the user may choose to surface treat one or both sides of the Virometer. Treating both sides of the device increases the likelihood that a VLP will adhere to one of the sides of the device because the average distance between a VLP and a charged surface is reduced.

When working with a fully sequenced and characterized virus, it is possible to infer what its IEP, measure the pH of its medium, and determine whether a positively or negatively charged device is appropriate. However, a more empirical strategy is required for unknown or heterogeneous virus samples. Factors that can influence a virus's charge include whether the virus is terrestrial or aquatic, the salinity of its medium, and the pH of its medium. If any of these elements are altered, it would be worthwhile to confirm the parameters that result in full VLP adhesion to the surface, as confirmed a lack of VLPs in the bulk. Furthermore, if an isolated sample has a lower concentration (˜10⁶ VLP/mL), it will require less dilution. This gives the user less control over the medium in which it resides, and it may be necessary to confirm that the virus readily sticks to the treated glass in its storage medium.

Initial counts for a new sample type should be compared to the Anodisc (or another established method) to ensure accuracy. If possible, technical replicates should be performed for both methods to compare the variability. The accuracy required of viral counts may vary based on the application. Based on the user's discretion, the Virometer can be fully adopted if the counts are repeatable/precise and within a range that investigators believe is similar enough to Anodisc counts. Otherwise more rigorous error analysis can be employed to determine what confidence interval would be tolerable relative to the Anodisc.

Overall sample processing also plays an important role in the quality of images obtained in the Virometer. DNase-treated samples will invariably be “cleaner” as there are no extraneous nucleic acids contaminating the sample and causing background noise to the mechanism that the fluorescent label employs. It is also important to ensure that all bacterial contamination has been removed from the sample. Viral concentrates must be diluted in virus-free water or buffer prior to imaging in the Virometer to achieve a concentration that can be resolved on the surface (<10⁹ VLP/mL), resulting in less debris and fewer autofluorescent salt deposits. Less processed raw samples are more prone to autofluorescent salt deposit accumulation, which adds noise to the images. A blank diluent/media control that has been treated with SYBR Gold II is necessary for each device to ensure that the media and the device itself are not contaminated with debris that could be counted as false positives. It is important to prevent virus aggregates forming after sample processing as these aggregates can result in undercounting the sample by as much as ten-fold. The aggregation that sometimes occurs at low temperatures (4° C.) has been observed to reverse itself at high temperatures (44° C.) in some cultured viruses. It is possible that aquatic viruses follow a similar trend, perhaps even recovering at lower temperatures since they are naturally found at lower temperatures compared to the cultured viruses analyzed by Lanni et al (Small Reversible Clumps of Bacteriophage and Their Anomalous Serologic Behavior. The Journal of Immunology. 1959 Aug. 1; 83 (2): 148-66). If VLP counts increase following a high temperature incubation, then it is likely that aggregates were present following low temperature storage. Although, there are multiple operating parameters to be considered when preparing samples in the Virometer, this provides the user with greater experimental flexibility as they are able to construct the Virometer in a way that is compatible with a wide array of sample types.

Another interesting avenue for the device would be multiplexing different fluorescent stains to characterize particle composition. Combining a membrane dye with a nucleic acid stain would allow the user to determine what proportion of EVs contain genetic material. Additionally, staining EVs with cell-specific fluorescently-tagged aptamers could provide insight into the cell(s) of origin of an EV population. This could have exciting ramifications in the context of cancer biomarkers.

Some viruses, such as T7, are dipoles and therefore weakly attracted to both surface treatments. However, some viruses are monopoles and exhibit a consistent charge across the entire capsid. The Virometer could serve as a tool to determine both the charge and the strength of that charge in a virus population. Virometers could be fabricated with a range of PLL concentrations and plasma treatment conditions to see which conditions facilitate virus adherence. Given that a virus's tendency to adhere to a surface fluctuates with temperature, pH, and salinity, it would be necessary to screen a wider range of buffers to determine which conditions do or do not lend themselves to virus adhesion. These experiments would be straightforward in homogeneous cultured virus samples and should therefore be validated in such systems. Furthermore, it could be possible to determine the isoelectric point (IEP) of a virus using the Virometer by keeping device surface charge, medium temperature, and medium salinity constant and altering the pH of the medium to determine the pH at which the virus changes from adhering to a positive surface to adhering to a negative surface. Viruses with known IEPs could be characterized in the Virometer to benchmark the IEP determined using this method against the IEP determined using other methods such as chromatofocusing or electrophoresis. Knowing the IEP of a virus allows the researcher to more easily determine what surface charge polarity and solution composition would result in optimal virus adhesion. Another benefit of the Virometer is that it offers user control of the environment that the virus is residing in, unlike chromatofocusing which requires the virus to be processed through a specialized column. This makes it possible to compare the behavior of water-bourne viruses in their native environment (temperature, pH, salinity) to artificial conditions on the bench top. In the case of heterogeneous environmental isolates, it would be important to differentiate the effects of two oppositely charged viruses from a single dipole.

The Virometer could also be used to enumerate viruses and their hosts at the same time. This presents some challenges because the host will invariably have a larger genome than the virus. This presents a problem when performing a nucleic acid stain because the signal will increase with genome size, causing the signal from the host to drown out the signal from the virus. One would also be forced to navigate possible differences in charge, although this could prove to be an advantage in separating viral populations for enumeration.

It may be possible to correlate genome size to fluorescence intensity in the Virometer. If this is the case, then it would have implications for sample evaluation in the context of sequencing. For example, it could serve as a secondary confirmation of whether the extracted nucleic acid originated from many viruses with small genomes or few viruses with large genomes. Genome size correlation would require rigorous calibration of fluorescent intensity based on viruses of known genome size.

Another possible avenue to pursue is sample retrieval. There may be cases in which a researcher wishes to retrieve their sample after imaging for additional downstream processing and analysis. It may be possible to flush the Virometer with a buffer with a pH that causes the virus to desorb from the surface resulting in viral release in a process analogous to selective elution from a purification column. Although the current Virometer design is incompatible with sample retrieval due to the staggered coverslip layering, assembling the device with a larger coverslip on the bottom and a smaller coverslip on top with double-sided tape spanning the entire width of the bottom coverslip would result in a lip on either end of the device that would allow the user to flush the chamber by pipetting buffer into one end while simultaneously withdrawing the displaced sample from the other end.

In a similar vein, the Virometer could conceivably be used as a means to archive virus samples. A benefit of the Anodisc is the ability to fix and freeze the sample for later analysis. The Virometer is currently being used as a disposable device that requires immediate epifluorescent imaging. However, it may be possible to fix and store samples in the Virometer for downstream super-resolution imaging. This could be achieved by treating the adhered sample with an appropriate fixative after loading into the modified design described in the previous paragraph, and then sealing the open ends of the sample chamber to prevent evaporation. Super-resolution techniques such as stimulated emission depletion (STED) microscopy, direct stochastic optical reconstruction microscopy (dSTORM), and photo-activation localization microscopy (PALM) have previously been applied to viruses such as HIV, influenza, and other human pathogens to probe architecture. It would be interesting to perform similar analyses on environmental samples and perhaps be able to characterize the morphological diversity of the viruses using a library of fluorescent tags.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for quantifying moving heterologous nanoparticles in a suspension by imaging, comprising:
(a) acquiring at least one z-stack of images within the suspension with an exposure time of 20-150 ms, wherein each z-stack of images consists of at least 5 images at an interval of 0.4-0.6 μm;
(b) tracking the nanoparticles in the images acquired in step (a) through x-axis, y-axis and z-axis of the suspension to identify unique nanoparticles; and
(c) enumerating the unique nanoparticles, wherein by the number of the nanoparticles in the suspension is obtained.

2. The method of claim 1, wherein step (b) comprises:
(i) identify non-overlapping nanoparticle tracks in each of the images acquired in step (a);
(ii) defining an exclusion zone around each of the non-overlapping nanoparticle tracks identified in step (i) that is equal to 20-30 nm in radius; and
(iii) excluding non-overlapping nanoparticle tracks that overlap with at least one of the exclusion zones defined in step (ii), whereby each remaining non-overlapping nanoparticle track corresponds to a unique nanoparticle.

3. The method of claim 1, further comprising determining the concentration of the nanoparticles in the suspension.

4. The method of claim 1, wherein the exposure time is 20-40 ms.

5. The method of claim 1, further comprising measuring a temperature.

6. The method of claim 1, wherein the nanoparticles are natural nanoparticles, synthetic nanoparticles, or a combination thereof.

7. The method of claim 1, wherein the nanoparticles are virus like particles (VLPs), and the suspension has a concentration of 10⁵-10⁹ VLPs/mL.

8. The method of claim 1, wherein the nanoparticles are labeled with a fluorescent agent.

9. The method of claim 1, wherein the nanoparticles carry nucleic acids labeled with an intercalating dye having a fluorescent intensity proportional to the amount of the nucleic acids, further comprising detecting the fluorescent intensity of the intercalating dye, and quantifying the amount of the nucleic acids based on the detected fluorescent intensity.

10. The method of claim 9, wherein the nanoparticles are viruses having a genome made of the nucleic acids, further comprising determining the size of the genome based on the amount of the nucleic acids and the number of the nanoparticles.

11. The method of claim 9, wherein the nanoparticles are extracellular vesicles.

12. The method of claim 1, wherein the nanoparticles comprise charged nanoparticles and non-charged nanoparticles, further comprising:
(d) placing the suspension in contact with a charged surface, whereby the charged nanoparticles are attached to the charged surface, and the non-charged nanoparticles remain in the suspension and are enumerated in step (c).

13. The method of claim 12, further comprising:
(e) acquiring images of a plurality of fields of view across the charged surface with an exposure time of 20-1000 ms;
(f) tracking the charged nanoparticles attached to the charged surface in the images acquired in step (e) to identify unique charged nanoparticles; and
(g) enumerating the unique charged nanoparticles attached to the charged surface.

14. The method of claim 13, further comprising determining a percentage of the charged nanoparticles based on the total number of the charged nanoparticles and the non-charged nanoparticles.

15. A method for characterizing size distribution of moving heterologous nanoparticles in a suspension by imaging, comprising:
(a) acquiring time lapse images at a minimum of 30 frames per second (fps) with an exposure time of 20-150 ms;
(b) tracking the nanoparticles in the images acquired in step (a) through x-axis, y-axis and z-axis of the suspension to identify unique nanoparticles;
(c) determining the locations of each of the unique particles;
(d) determining the size of each of the unique nanoparticles based on the locations in step (c); and
(e) aggregating the sizes of the unique nanoparticles in step (d), whereby the size distribution of the nanoparticles in the suspension is characterized.

16. The method of claim 15, wherein step (b) comprises:
(i) identify non-overlapping nanoparticle tracks in each of the images acquired in step (a);
(ii) defining an exclusion zone around each of the non-overlapping nanoparticle tracks identified in step (i) that is equal to 20-30 nm in radius; and
(iii) excluding non-overlapping nanoparticle tracks that overlap with at least one exclusion zones defined in step (ii), whereby each remaining non-overlapping nanoparticle track corresponds to a unique nanoparticle.

17. The method of claim 15, wherein step (c) comprises:
(iv) determining the location of each unique nanoparticle based on the average position in the x-axis, y-axis and z-axis of all points in the corresponding non-overlapping track.

18. The method of claim 1, wherein step (d) comprises:
(v) determining diffusion coefficient (D) for each unique nanoparticle using Equation 1:
$${Δ}_{ms} = 4 D Δ t \qquad Equation 1$$
wherein Δₘₛ is a mean squared displacement and Δt is a duration of a frame; and
(vi) determining a diameter (d) of each unique nanoparticle using Equation
$$d = \frac{{k}_{b} T}{3 πη D} \qquad Equation 2$$
wherein D is diffusion coefficient, T is temperature, η is medium viscosity, and k_{b} is the Boltzmann constant.

19. The method of claim 15, wherein the time lapse images are acquired at an intensity of 5-25%.

20. The method of claim 15, wherein the time lapse images are acquired at an exposure time of 20-40 ms.

21. The method of claim 15, wherein at least 30 frames of images are acquired during each exposure time.

22. The method of claim 15, further comprising measuring temperature.

23. The method of claim 15, wherein the nanoparticles are natural nanoparticles, synthetic nanoparticles, or a combination thereof.

24. The method of claim 15, wherein the nanoparticles are virus like particles (VLPs), and the suspension has a concentration of 10⁵-10⁹ VLPs/mL.

25. The method of claim 15, wherein the nanoparticles are labeled with a fluorescent agent.

26. The method of claim 15, wherein the nanoparticles carry nucleic acid labeled with an intercalating dye having a fluorescent intensity proportional to the amount of the nucleic acid, further comprising detecting the fluorescent intensity of the intercalating dye, and quantifying the amount of the nucleic acid based on the detected fluorescent intensity.

27. The method of claim 26, wherein the nanoparticles are viruses having a genome made of the nucleic acid, further comprising determining the size of the genome based on the amount of the nucleic acid and the number of the nanoparticles.

28. The method of claim 26, wherein the nanoparticles are extracellular vesicles.

29. The method of claim 15, wherein the nanoparticles comprise charged nanoparticles and non-charged nanoparticles, further comprising:
(f) placing the suspension on a charged surface, whereby the charged nanoparticles are attached to the charged surface, and the non-charged nanoparticles remain in the suspension and characterized for size distribution in step (e).

30. The method of claim 29, further comprising:
(g) acquiring images of a plurality of fields of view across the charged surface with an exposure time of 20-1000 ms;
(h) tracking the charged nanoparticles in the images acquired in step (e) to identify unique charged nanoparticles; and
(i) enumerating the unique charged nanoparticles.
